(19) United States
(12) Patent Application Publication
(10) Pub. No.: US 2025/0281929 A1
(43) Pub. Date: Sep. 11, 2025

(54) MICROFLUIDIC DEVICE AND USES THEREOF

(71) Applicant: CASE WESTERN RESERVE UNIVERSITY, Clevland, OH (US)

(72) Inventors: UMUT GURKAN, Cleveland, OH (US); DEBNATH MAJI, Cleveland, OH (US); PEDRAM MOHSENI, Cleveland, OH (US); MICHAEL SUSTER, Cleveland, OH (US); YUCHENG MAN, Cleveland, OH (US)

(21) Appl. No.: 18/265,087

(22) PCT Filed: Dec. 3, 2021
(86) PCT No.: PCT/US2021/061757

§ 371 Date: Jun. 2, 2023

Related U.S. Application Data
(60) Provisional application No. 63/121,045, filed on Dec. 3, 2020.

(51) Int. Cl. B01L 3/00

(52) U.S. Cl. CPC B01L 3/502753; B01L 2300/048; B01L 2300/0636; B01L 2300/0645; B01L 2300/0809; B01L 2300/12

(57) ABSTRACT

A microfluidic device includes at least one microchannel with a plurality of micropillar arrays and pairs of electrodes flanking each micropillar array along a length of the microchannel. Each micropillar array defines a plurality of microcapillaries having a separation distance, and the separation distance of the microcapillaries defined by each micropillar array decreases in a direction of fluid flow through the microchannel. The pairs of electrodes are configured to measure electrical impedance in each respective micropillar array.

### RELATED APPLICATION

This application claims priority from U.S. Provisional Application No. 63/121,045, filed Dec. 3, 2020, the subject matter of which is incorporated herein by reference in its entirety.

### GOVERNMENT FUNDING

This invention was made with government support under 1552782 awarded by the National Science Foundation and HL133574 and OT2HL152643 awarded by the National Heart, Lung, and Blood Institute of the National Institutes of Health.” The government has certain rights in the invention.

### BACKGROUND

Deformability of red blood cells (RBCs) is critical for continuous blood flow in the microcirculation. Healthy RBCs are highly deformable, allowing them to pass through minuscule blood vessels to facilitate oxygen delivery. However, in RBC disorders, such as sickle cell disease (SCD) and malaria, deformability is pathologically altered. In SCD, abnormal RBC deformability is induced by intracellular polymerization of sickle hemoglobin (HbS) under deoxygenated conditions. Impaired RBC deformability significantly contributes to the pathophysiological hallmarks of this disorder, including hemolysis, inflammation, microvascular occlusion and consequent organ failures. In malaria, aberrant RBC deformability is caused by membrane-protein network modifications associated with Plasmodium parasites. Conventional techniques developed for RBC deformability assessment include atomic force microscopy, micropipette aspiration, optical tweezers, and osmotic gradient ektacytometry. Even though these techniques have proven useful in assessing RBC deformability in vitro, they are time and skill intensive, low-throughput, can exhibit limited physiological relevance and clinical utility.

Microfluidic technologies have been developed to better recapitulate in vivo microvascular environment and to probe RBC deformability in precisely controlled flow conditions at both bulk and single-cell level. Single-cell microfluidic approaches have been used to measure RBC deformability by assessing micro-constriction obstruction, shear deformation, membrane fluctuation, lateral margination, cell velocity, transient time, electrical impedance, and electrical deformation. Single-cell approaches are low-throughput and yield limited information on a small fraction of cells, which may not represent the overall cell population. Bulk-cell microfluidic approaches have been utilized to characterize the deformability of bulk RBCs by measuring the average cell retention, cell elongation, and average cell transient velocity. However, none of these techniques fully mimic the capillary bed architecture or provide a direct assessment of the pathophysiological impact of impaired RBC deformability on the microcirculation.

### SUMMARY

This disclosure describes a microfluidic device and system for measuring vascular occlusion and/or cell adhesion, and particularly relates to a microfluidic device and system for microvascular occlusion rate associated with red blood cell (RBC) deformability and adhesion rate associated with RBC deformability and adhesion. In some embodiments, the microfluidic device can mimic architectural features associated with capillary beds of a subject. The microfluidic device can include a plurality of micropillar arrays within a microchannel that define a plurality of microcapillaries that mimic capillary networks of microvasculature of a subject and integrated electrical impedance measurement electrodes to assess RBC-mediated occlusion within the plurality of microcapillaries defined by the micropillar arrays. These microcapillaries can be engineered to retain RBCs with impaired deformability, such that more abnormal RBCs will occlude wide upstream microcapillaries, while those with moderate impairment will occlude finer downstream microcapillaries within the microchannel. The microchannel can also be designed with two wide side or outer openings or passages to mimic arteriovenous anastomoses, which act as shunts in the capillary bed in vivo. These side anastomoses can prevent complete blockade of flow in the microchannel, and enable testing of clinical blood samples with near-physiological hematocrit levels.

Advantageously, RBCs perfused through the microfluidic device experience a wide spectrum of deformations when crossing microcapillaries with different sizes, which recapitulates a more physiologically relevant microenvironment. Additionally, the microfluidic device can examine large numbers of heterogeneous RBCs since the embedded micropillar arrays recapitulate large numbers of microcapillaries with various dimensions, enabling the simultaneous deformability analysis of bulk RBCs at a single-cell level. Moreover, the integrated electrical impedance measurement provides a reproducible functional measurement for standardized assessment of RBC-mediated microvascular occlusion associated with abnormal RBC deformability and an in vitro therapeutic efficacy benchmark for assessing clinical outcome of emerging RBC modifying targeted and curative therapies. The microfluidic device provides a finer and more rapid detection compared to previous devices and an alternative functional metric for standardized measurements of RBC mediated microvascular occlusion without need for imaging. Furthermore, the microfluidic device can be used to assess very small changes in RBC deformability, under both normoxic (ambient air) and hypoxic conditions, to assess pathologically impaired RBCs in blood. The assessment of pathologically impaired RBCs can be used to assess microvascular health and function of a subject and determine the subject's increased risk of vaso-occlusive crises (VOC) in a range of microcirculatory diseases.

In some embodiments, the microfluidic device can include at least one microchannel that extends through a portion of a housing. The at least one microchannel can be configured to receive a fluid sample that flows along a length of the microchannel from a first end to a second end of the microchannel. The at least one microchannel can include a plurality of micropillar arrays provided along the length of the microchannel and pairs of electrodes positioned on opposite sides of each micropillar array in a direction of fluid flow through the microchannel. Each micropillar array can define a plurality of microcapillaries having a separation distance, and the separation distance of the microcapillaries defined by each micropillar array decreases in a direction of fluid flow through the microchannel. The pairs of electrodes are configured to measure electrical impedance in each respective micropillar array.

In some embodiments, the microchannel can include a substantially planar upper surface and a substantially planar lower surface. The micropillars of the plurality of micropillar arrays can extend from upper surface to the lower surface.

In some embodiments, each of the micropillars of the plurality of micropillar arrays can have a substantially rectangular cross section.

In other embodiments, each of the microcapillaries can have a substantially rectangular cross section.

In some embodiments, the microchannel includes at least three micropillar arrays and the separation distances of the microcapillaries defined by each respective micropillar array are substantially uniform.

In other embodiments, each micropillar array includes at least three rows of micropillars. The rows can extend perpendicular to fluid flow and have a substantially similar shape.

In some embodiments, the distance between each micropillar in a row of a respective micropillar array is substantially the same.

In some embodiments, successive micropillar arrays are separated from each other in the microchannel by a gap region. The gap region can be free of micropillars and each gap region can include an electrode. Electrodes in successive gap regions can be configured to measure electrical impedance in micropillar arrays flanked by the gap regions

In some embodiments, the microchannel can include a first micropillar array at the first end that defines a plurality of first microcapillaries that each have a separation distance of about 12 μm. Each successive micropillar array in the direction of fluid flow through the microchannel can define a plurality of microcapillaries that each have a separation distance of about 10% to about 30% less than a plurality of microcapillaries defined by a preceding micropillar array.

In other embodiments, the microchannel includes a micropillar array at the second end that defines a plurality of microcapillaries that each have a separation distance of about 3 μm. Each preceding micropillar array in a direction opposite the direction of fluid flow through the microchannel can define a plurality of microcapillaries that each have a separation distance of about 20% to about 50% greater than a plurality of microcapillaries defined by a preceding micropillar array.

In some embodiments, the separation distance of the plurality of microcapillaries defined by at least one of the plurality of micropillar arrays permits passage of healthy cells in a fluid sample perfused through the microchannel but is occluded by cells with impaired deformability.

In some embodiments, the fluid sample includes blood cells, such as RBCs. The fluid sample can include blood with at least about 1%, at least about 5%, at least about 10%, at least about 15%, at least about 20%, at least about 30%, or at least about 40% hematocrit.

In some embodiments, the separation distance of the plurality of microcapillaries at the second end of the microchannel can be such that the microcapillaries can be occluded by abnormal RBCs in a fluid sample perfused through the microchannel.

In other embodiments, each of the micropillar arrays can be arranged in an inner portion of the microchannel that extends the length of the microchannel. The microchannel can include two parallel outer passages on opposite sides of the inner portion that extend the length of the microchannel. The outer passages can be in fluid communication with the plurality of microcapillaries defined by the plurality of micropillar arrays. The outer passages can have separation distances or cross sectional areas that permit cells in a fluid sample to flow through the microchannel without being occluded and/or obstructed.

In some embodiments, the electrodes are planar and are provided on a lower surface channel of the microchannel. The electrodes can be uniformly spaced along the microchannel.

In some embodiments, the measured impedance is indicative of RBC-mediated microvascular occlusion in the microcapillaries of the respective micropillar array.

In some embodiments, the microchannel includes a substantially planar transparent wall that defines the upper surface or lower surface of the microchannel. The substantially planar transparent wall can permit observation into the microfluidic channel by microscopy.

In some embodiments, the microfluidic device can further include a micro-gas exchanger and a chamber for controlling the oxygen content of the fluid sample prior to and/or after delivering the fluid sample to the at least one microchannel. The micro-gas exchanger can provide hypoxic blood to the at least one microchannel.

In other embodiments, the microfluidic device can include at least one capturing agent that is immobilized on a surface of the at least one microchannel. The capturing agent can adhere a cell of interest to the at least one surface of the at least one microchannel when a fluid sample containing cells is passed through the at least one microchannel. The at least one capturing agent can include, for example, at least one of laminin, fibronectin, E-Selectin, P-Selectin, L-selectin, intracellular adhesion molecule 1 (ICAM-1), or vascular cellular adhesion molecule 1 (VCAM-1).

Other embodiments described herein relate to a microfluidic system comprising the microfluidic device described herein.

The microfluidic system can include a pressure pump and a reservoir that are in fluid communication with the at least one microchannel of the microfluidic device. The reservoir can include a fluid sample that includes blood cells. The pressure pump is configured to provide pressure to the reservoir such that the fluid sample flows through the at least one microchannel.

In some embodiments, the fluid sample can flow through the at least one microchannel at a physiologically relevant flow velocity. The physiologically relevant flow velocity can be about 1 mm/s to about 2 mm/s.

In some embodiments, the microfluidic system can include an impedance measuring system for measuring electrical impedance between pairs of electrodes flanking respective micropillar arrays. The impedance measuring system can include a processor configured to compare and/or determine changes of measured impedances of pairs of electrodes.

In some embodiments, the processor can determine cumulative impedance changes of pairs of electrodes flanking the at least one micropillar array by comparing the impedance of pairs of electrodes before and after a blood sample is perfused through the microchannel.

In some embodiments, each micropillar array can define a capillary network and the processor can be configured to determine a red blood cell (RBC) Electrical Impedance Index (REI) of a blood sample perfused through the microchannel of the microfluidic device based on the following equation:

$$REI = {\sum }_{i}^{n} \frac{{I}_{i 2} - {I}_{i 1}}{{I}_{i 1}} \times 100 \%$$

where,
n=total number of capillary networks within an area of interest;
i=the i^{th} capillary network within the area of interest;
Iᵢ₁=a first electrical impedance reading of an i^{th} capillary network; and
Iᵢ₂=a second electrical impedance reading of the i^{th} capillary network.

The REI can be indicative of RBC-mediated occlusion of microcapillaries in the microchannel.

In some embodiments, the processor can provide the same weight to electrical impedances measured from different pairs of electrodes. For example, the measured impedance of a pair of electrodes upstream in the microchannel can be weighted the same as a pair of electrodes downstream in the microchannel. The impedance measurements can be cumulated and compared to control impedance measurements to provide the cumulative percentage impedance changes.

In other embodiments, the microfluidic system can further include an imaging system for measuring the deformability, adherence, and/or number of cells of interest in the at least one microchannel when the fluid sample is passed therethrough. The imaging system can measure the number of occluded microcapillaries in the at least one microchannel. The imaging system includes a control unit for determining viscosity of the fluid sample. The viscosity of the fluid sample can be determined by measuring the mean flow velocity of the fluid sample as it passes through the microchannel.

Other embodiments described herein relate to a method of assessing the pathology of RBCs. The method can include providing a microfluidic device as described herein and measuring a first impedance of at least one micropillar array before a fluid sample including RBCs is perfused through the at least one microchannel. A fluid sample including the RBCs can then be perfused through the at least one microchannel of the microfluidic device. A second impedance of the at least one micropillar array can be measured after perfusion of the fluid sample through the microchannel. The RBC pathology can be determined by comparing the first impedance and the second impedance.

In some embodiments, the first impedance is measured while phosphate buffered saline (PBS) is perfused through the microchannel.

In other embodiments, PBS can be perfused through the microchannel after perfusing the fluid sample but before the second impedance measurement.

In some embodiments, a cumulative impedance change of pairs of electrodes flanking at least one micropillar array can be calculated.

In some embodiments, a red blood cell (RBC) Electrical Impedance Index (REI) of the fluid sample perfused through the microchannel of the microfluidic device based on the following equation:

$$REI = {\sum }_{i}^{n} \frac{{I}_{i 2} - {I}_{i 1}}{{I}_{i 1}} \times 100 \%$$

wherein each micropillar array defines a capillary network;
n=total number of capillary networks within an area of interest;
i=the i^{th} capillary network within the area of interest;
Iᵢ₁=a first electrical impedance reading of an i^{th} capillary network; and
Iᵢ₂=a second electrical impedance reading of the i^{th} capillary network
wherein the REI is indicative of a RBC pathology or RBC-mediated occlusion of microcapillaries in the microchannel.

In other embodiments, the impedance of each capillary network can be weighted the sane relative to other capillary networks in determining Iᵢ₁ and Iᵢ₂.

In some embodiments, the RBCs are from a subject at risk of vaso-occlusive crises and/or decreased microvascular health and function. The subject can have an increased risk of vaso-occlusive crises (VOC) and/or decreased microvascular health and function when the REI is greater than a control value.

In some embodiments, the fluid sample includes at least about 1%, at least about 5%, at least about 10%, at least about 15%, or at least about 20%, at least about 30%, or at least about 40% hematocrit. The RBCs can be from stored blood and/or blood to be transfused. The fluid sample can also be whole blood obtained from a subject.

In some embodiments, the first impedance and the second impedance are measured at a frequency of about 40 Hz to about 1 MHz, about 100 Hz to about 100 kHz, or about 1 kHz to about 50 kHz.

In other embodiments, the fluid sample can be perfused under at least one of normoxic or hypoxic conditions.

In some embodiments, the fluid sample is a blood sample obtained from a subject having or at increased risk of microvascular occlusion, sickle cell disease, malaria, or in need of transfusion. The subject in need of transfusion can have or be at risk of hereditary spherocytosis, iron-deficiency anemia, aplastic anemia, Crohn's disease, systemic lupus, rheumatoid arthritis, thalassemia, multiple myeloma, leukemia, hemophilia, and Von Willebrand disease.

Other embodiments described herein relate to a method of measuring efficacy of a therapeutic agent in modulating blood cell adhesion and/or deformability and/or microvascular occlusion. The method can include provide a microfluidic device as described herein and perfusing a fluid sample including blood cells through the at least one microchannel of the microfluidic device. The impedance of at least one micropillar array in the at least one microchannel can be measured after the fluid sample is perfused through the microchannel. The therapeutic agent can be added to at least one of the fluid sample prior to perfusion through the at least one microchannel. The efficacy of the therapeutic agent can be determined based on the measured impedance of the at least one micropillar array. A decrease in the measured impedance compared to a control is indicative of the therapeutic agent having an increased efficacy in decreasing blood cell adhesion and/or increasing blood cell deformability.

In some embodiments, the fluid sample can be perfused under at least one of normoxic or hypoxic conditions.

Other embodiments described herein relate to a method of assessing clinical outcome of a subject administered a RBC therapy. The method can include providing a microfluidic device as described herein and perfusing a fluid sample including RBCs from the subject administered the RBC therapy through the at least one microchannel of the microfluidic device. The impedance of at least one micropillar array in the at least one microchannel can be measured after the fluid sample is perfused through the microchannel. The clinical outcome of the subject can be determined by comparing the measured impedance of the at least one micropillar array to a control.

In some embodiments, a difference in the measured impedance compared to a control is indicative of a favorable clinical outcome.

In other embodiments, a difference in the measured impedance compared to a control is indicative of an unfavorable clinical outcome.

### BRIEF DESCRIPTION OF THE DRAWINGS

FIG. 1 illustrates a schematic drawing of a microfluidic system in an accordance with an embodiment described herein.

FIG. 2 illustrates a schematic drawing of a microfluidic device in accordance with an embodiment described herein. Arrows indicated direction of flow

FIG. 3 illustrates a schematic of a plurality of micropillars and electrodes in a microchannel of the microfluidic device of FIG. 2. Arrows indicated direction of flow.

FIG. 4 illustrates a schematic of fluid flow through a row a micropillar array of a microchannel. Arrows indicate direction of flow.

FIG. 5 is a flow diagram of a method in accordance with an embodiment described herein.

FIGS. 6(A-D) illustrate schematic drawings, images, and a plot showing microfluidic electrical impedance assessment of red blood cell (RBC) mediated microvascular occlusion. (A) Electrical impedance across two electrodes placed on either side of the micropillar array is measured before and after sample perfusion. The resultant impedance change depends on microcapillary occlusion in the array. (B) The microfluidic device consists of six micropillar arrays comprising microcapillaries from 12 μm down to 3 μm, which mimic key dimensions of small blood vessels observed in the capillary bed. The 12-μm array is designed to trap potential large-cell aggregates that may cause microchannel clogging. Inset: Schematic of the capillary-inspired micropillar array. The 40-μm-wide side pathway is designed to mimic arteriovenous anastomoses to help regulate flow and to prevent upstream clogging. Schematics are not drawn to scale, and all dimensions are in microns. (C) Photograph of the microfluidic device is shown. Arrow indicates flow direction. Inset: Close-up views of the microcapillary occlusion within the 3-μm array induced by glutaraldehyde-stiffened RBCs or healthy RBCs. (D) Temporal variation in electrical impedance of the 3-μm array observed at 10 kHz is shown for a sample with 2% stiff RBCs and 98% healthy RBCs, a sample with 100% healthy RBCs, and PBS. Each sample was perfused for 20 min, which was followed by post-perfusion wash with PBS for another 20 min. The initial impedance reading was taken at the start point of each test (Time=0 min), and the second impedance reading was taken at the endpoint (Time=40 min).

FIGS. 7(A-E) illustrate graphs and a plot showing system characterization using glutaraldehyde-stiffened RBCs (stiff RBCs) and analysis of the microcapillary occlusion and impedance data. (A) Profiles of microcapillary occlusion in the 3-μm to 10-μm arrays shown as histograms for the tested RBC samples with 100% healthy RBCs, with 99% healthy RBCs and 1% stiff RBCs, or with 98% healthy RBCs and 2% stiff RBCs. (B) The ROI of samples with 98% healthy RBCs and 2% stiff RBCs was significantly higher compared to that of samples with 99% healthy RBCs and 1% stiff RBCs or 100% healthy RBCs, and the ROI of samples with 99% healthy RBCs and 1% stiff RBCs was significantly higher compared to that of 100% healthy RBCs (p<0.05, paired t-test). (C) Profiles of impedance change in those arrays shown as histograms for the tested RBC samples. (D) The REI of samples with 98% healthy RBCs and 2% stiff RBCs was significantly higher compared to that of samples with 99% healthy RBCs and 1% stiff RBCs or 100% healthy RBCs, and the REI of samples with 99% healthy RBCs and 1% stiff RBCs was significantly higher compared to that of 100% healthy RBCs (p<0.05, paired t-test). (E) The REI significantly correlates with the ROI in the tested samples (PCC=0.987, p<0.0001, N=12). ROI: RBC Occlusion Index. REI: RBC Electrical Impedance Index. PCC: Pearson correlation coefficient. Error bars represent standard deviation. N=4 for each group.

FIG. 8 illustrates a graph showing the measurement reproducibility was assessed by repeatedly testing one RBC sample from a single healthy donor using five different devices. Shown are the ROI and REI results of the five repeats (mean±standard deviation). ROI: RBC Occlusion Index. REI: RBC Electrical Impedance Index. Error bars represent standard deviation.

FIGS. 9(A-E) illustrate graphs and a plot showing assessment of RBC deformability and microvascular occlusion in two common red cell disorders, sickle cell disease (SCD) and hereditary spherocytosis (HS). (A) Histograms of microcapillary occlusion for the tested RBC samples from healthy donors and subjects with SCD or HS. (B) The ROI of RBCs from subjects with SCD or HS is significantly higher compared to that from healthy donors (p<0.05, one-way ANOVA). (C) Histograms of impedance change for the tested RBC samples from healthy donors and subjects with SCD or HS. (D) The REI of RBCs from subjects with SCD or HS is also significantly higher compared to that from healthy donors (p<0.05, one-way ANOVA). (E) The REI significantly correlates with the ROI in the tested samples (PCC=0.946, p<0.0001, N=19). ROI: RBC Occlusion Index. REI: RBC Electrical Impedance Index. PCC: Pearson correlation coefficient. Error bars represent standard deviation. N=5 for healthy, N=12 for SCD, and N=2 for HS.

FIGS. 10(A-E) illustrate a plot and graphs showing the microfluidic electrical impedance assessment of RBC mediated microvascular occlusion as an in vitro therapeutic efficacy benchmark to assess the clinical outcome of treatments in SCD. (A) A subpopulation (Group 1, N=5) with distinct ROI and REI profiles compared to the rest (Group 2, N=7) was identified. Group 1 subjects had significantly lower (B) ROI (p<0.001, one-way ANOVA) and (C) REI (p=0.006, Mann-Whitney) compared to Group 2 subjects. Moreover, Group 1 subjects had relatively lower (D) serum lactate dehydrogenase (LDH) levels (p=0.052, Mann-Whitney) and (E) absolute reticulocyte counts (ARCs) (p=0.037, Mann-Whitney) compared to Group 2 subjects. The dashed rectangular regions represent typical normal ranges for the given clinical parameters. HSCT: hematopoietic stem-cell transplantation. ROI: RBC Occlusion Index. REI: RBC Electrical Impedance Index. Error bars represent standard deviation.

FIG. 11 is a schematic illustration of fabrication process of the microfluidic device. The gold electrodes were initially patterned under a Kapton tape mask on a standard microscope glass side, after which the SiO₂ was deposited under a secondary Kapton tape mask. Finally, PDMS was covalently bonded to the substrate. The cross marks were designed for micropillar array and gold electrode alignment. The titanium adhesion layer between the glass and gold is not shown.

FIG. 12 illustrates a schematic illustration of an experimental setup. The RBC sample at 20% hematocrit is loaded in the sample reservoir and perfused through the microchannel for 20 min at 100 mBar using a Fluigent Flow-EZ pump with positive pressure. An impedance analyzer is connected to the device contact pads using electrical wires for impedance recording. The microfluidic device is mounted on the stage of an inverted microscope for image recording.

FIG. 13 illustrates a plot showing the raw impedance data across the 3-μm micropillar array measured for a clinical blood sample. Magnitude of impedance across the micropillar array was recorded over the frequency range of 40 Hz-1 MHz before introducing blood into the microchannel (Solid Line) and after completing the washing step (Square Markers). Percentage change of impedance was analyzed at 10 kHz.

FIG. 14 illustrate a graph showing the profiles of impedance change in the 3-μm to 10-μm micropillar arrays for testing PBS using 5 devices from different manufacturing batches. The 95% confidence interval of the impedance change data was determined as 0.01-0.27%, from which the background noise was determined. Error bars represent the standard deviation.

FIGS. 15(A-D) illustrate plots showing the ROI and REI significantly correlate with in vivo hemolytic biomarkers in the study subjects with SCD. (A) The ROI significantly correlates with (A) serum lactate dehydrogenase (LDH) levels (PCC=0.814, p=0.001, N=12) and (B) absolute reticulocyte counts (ARCs) (PCC=0.582, p=0.047, N=12). Similarly, the REI significantly correlates with (C) serum LDH levels (PCC=0.698, p=0.002, N=12) and (D) ARCs (PCC=0.731, p=0.007, N=12). ROI: RBC Occlusion Index. REI: RBC Electrical Impedance Index. PCC: Pearson correlation coefficient.

FIGS. 16(A-B) illustrate graphs showing subjects with SCD on-transfusion (N=8) had significantly higher (A) ROI (p=0.021, one-way ANOVA) and (B) REI (p=0.022, one-way ANOVA) compared to healthy donors (N=5). ROI: RBC Occlusion Index. REI: RBC Electrical Impedance Index. Error bars represent standard deviation.

FIGS. 17(A-B) illustrate graphs showing no significance was observed in (A) ROI or (B) REI between females (N=7) and males (N=5) over the study population with SCD. ROI: RBC Occlusion Index. REI: RBC Electrical Impedance Index. Error bars represent standard deviation.

FIG. 18 illustrates RBC mediated microcapillary occlusion measured by a microfluidic device and the newly defined Occlusion Index (OI) associates with clinical phenotype in SCD. (′N′ represents the number of subjects tested). The hypothesized levels following an effective therapy, with reversal of the abnormal red cell phenotype RBCs from sickle cell trait (HbAS) subjects induced significantly greater OI values, under hypoxia compared to normoxia (N=5), similar to those from sickle cell variant (HbSC) subjects (N=3) or HbSS subjects (N=4). RBCs from HbAA subjects (healthy controls) induced negligible OI under either normoxia or hypoxia (N=3). * Denotes a statistical significance.

### DETAILED DESCRIPTION

To facilitate the understanding of this invention, a number of terms are defined below. Terms defined herein have meanings as commonly understood by a person of ordinary skill in the areas relevant to the present invention. Terms such as “a”, “an”, and “the” are not intended to refer to only a singular entity but also plural entities and also includes the general class of which a specific example may be used for illustration. The terminology herein is used to describe specific aspects of the invention, but their usage does not delimit the invention, except as outlined in the claims.

Throughout the description, where compositions are described as having, including, or comprising, specific components, it is contemplated that compositions also consist essentially of, or consist of, the recited components. Similarly, where methods or processes are described as having, including, or comprising specific process steps, the processes also consist essentially of, or consist of, the recited processing steps. Further, it should be understood that the order of steps or order for performing certain actions is immaterial so long as the compositions and methods described herein remains operable. Moreover, two or more steps or actions can be conducted simultaneously.

As used herein, the term “about” or “approximately” refers to a quantity, level, value, number, frequency, percentage, dimension, size, amount, weight or length that varies by as much as 15%, 10%, 9%, 8%, 7%, 6%, 5%, 4%, 3%, 2% or 1% to a reference quantity, level, value, number, frequency, percentage, dimension, size, amount, weight or length. In one embodiment, the term “about” or “approximately” refers a range of quantity, level, value, number, frequency, percentage, dimension, size, amount, weight or length±15%, ±10%, ±9%, ±8%, ±7%, ±6%, ±5%, ±4%, ±3%, ±2%, or ±1% about a reference quantity, level, value, number, frequency, percentage, dimension, size, amount, weight or length.

It is further noted that the claims may be drafted to exclude any optional element. As such, this statement is intended to serve as antecedent basis for use of such exclusive terminology as “solely”, “only” and the like in connection with the recitation of claim elements, or the use of a “negative” limitation. “Optional” or “optionally” means that the subsequently described circumstance may or may not occur, so that the description includes instances where the circumstance occurs and instances where it does not.

The term “microchannels” as used herein refer to pathways through a medium, e.g., silicon, that allow for movement of liquids and gasses. Microchannels can therefore connect other components, i.e., keep components “in fluid communication.” While it is not intended that the present application be limited by precise dimensions of the channels, illustrative ranges for channels are as follows: the channels can be between 0.1 and 100 μm in depth (e.g., 50 μm) and between 50 and 10,000 μm in width (e.g., 400 μm). The channel length can be between 1 mm and 100 mm (e.g., about 27 mm).

The term “microfabricated”, “micromachined”, and/or “micromanufactured” as used herein means to build, construct, assemble or create a device on a small scale, e.g., where components have micron size dimensions or microscale.

The term “polymer” as used herein refers to a substance formed from two or more molecules of the same substance. Polymers may also be linear polymers in which the molecules align predominately in chains parallel or nearly parallel to each other. In a non-linear polymer, the parallel alignment of molecules is not required.

The term “lensless image” or “lensless mobile imaging system” as used herein refers to an optical configuration that collects an image based upon electronic signals as opposed to light waves. For example, a lensless image may be formed by excitation of a charged coupled device (CCD) sensor by emissions from a light emitting diode.

The term “charge-coupled device (CCD)” as used herein refers to a device for the movement of electrical charge, usually from within the device to an area where the charge can be manipulated, for example, a conversion into a digital value. A CCD provides digital imaging when using a CCD image sensor where pixels are represented by p-doped MOS capacitors.

The term “symptom” as used herein refers to any subjective or objective evidence of disease or physical disturbance observed by the patient. For example, subjective evidence is usually based upon patient self-reporting and may include, but is not limited to, pain, headache, visual disturbances, nausea, and/or vomiting. Alternatively, objective evidence is usually a result of medical testing including, but not limited to, body temperature, complete blood count, lipid panels, thyroid panels, blood pressure, heart rate, electrocardiogram, tissue, and/or body imaging scans.

The term “disease” or “medical condition”, as used herein, refers to any impairment of the normal state of the living animal that interrupts or modifies the performance of the vital functions. Typically manifested by distinguishing signs and symptoms, it is usually a response to: i) environmental factors (as malnutrition, industrial hazards or climate); ii) specific infective agents (as worms, bacteria or viruses); iii) inherent defects of the organism (as genetic anomalies); and/or iv) combinations of these factors.

The term “patient” or “subject” as used herein is a human or animal and need not be hospitalized. For example, out-patients, persons in nursing homes are “patients.” A patient may comprise any age of a human or non-human animal and therefore includes both adult and juveniles, i.e., children. It is not intended that the term “patient” connote a need for medical treatment and, thus, a patient may voluntarily or involuntarily be part of experimentation whether clinical or in support of basic science studies.

The term “functionalized” or “chemically functionalized” as used herein means the addition of functional groups onto the surface of a material by chemical reaction(s). As will be readily appreciated by a person skilled in the art, functionalization can be employed for surface modification of materials in order to achieve desired surface properties, such as biocompatibility, wettability, and so on. Similarly, the term “biofunctionalization,” “biofunctionalized,” or the like, as used herein, means modification of the surface of a material to have desired biological function, which will he readily appreciated by a person of skill in the related art, such as bioengineering.

The term “sample” as used herein is used in its broadest sense and includes environmental and biological samples. Environmental samples include material from the environment such as soil and water. Biological samples may be animal, including, human, fluid, e.g., blood, plasma, and serum; solid, e.g., stool; tissue; liquid foods, e.g., milk; and solid foods, e.g., vegetables. A biological sample may comprise a cell, tissue extract, body fluid, chromosomes or extrachromosomal elements isolated from a cell, genomic DNA (in solution or bound to a solid support such as for Southern blot analysis), RNA (in solution or bound to a solid support such as for Northern blot analysis), cDNA (in solution or bound to a solid support) and the like.

The terms “capturing agent”, “bioaffinity ligand”, “binding component”, “ligand” or “receptor” as used herein may be any of a large number of different molecules, biological cells or aggregates, and the terms are used interchangeably. Each capturing agent may be immobilized on a solid substrate and binds to an analyte being detected. Proteins, polypeptides, peptides, nucleic acids (nucleotides, oligonucleotides and polynucleotides), antibodies, ligands, saccharides, polysaccharides, microorganisms such as bacteria, fungi, and viruses, receptors, antibiotics, test compounds (particularly those produced by combinatorial chemistry), plant and animal cells organdies or fractions of each and other biological entities may each be a capturing agent. Each, in turn, also may be considered as analytes if same bind to a capturing agent.

The terms “bind” or “adhere” as used herein include any physical attachment or close association, which may be permanent or temporary. Generally, an interaction of hydrogen bonding, hydrophobic forces, van der Waals forces, covalent and ionic bonding etc., facilitates physical attachment between the capturing agent and the analyte being measured. The “binding” interaction may be brief as in the situation where binding causes a chemical reaction to occur. That is typical when the binding component is an enzyme and the analyte is a substrate for the enzyme. Reactions resulting from contact between the capturing agent and the analyte are also within the definition of binding for the purposes of this application.

The term, “substrate” as used herein refers to surfaces as well as solid phases, which may include a microchannel. In some cases, the substrate is solid and may comprise PDMS. A substrate may also include components including, but not limited to, glass, silicon, quartz, plastic or any other composition capable of supporting photolithography.

The term, “photolithography”, “optical lithography” or “UV lithography” as used herein refers to a process used in microfabrication to pattern parts of a thin film or the bulk of a substrate. It uses light to transfer a geometric pattern from a photomask to a light-sensitive chemical “photoresist” or simply “resist,” on the substrate. A series of chemical treatments then either engraves the exposure pattern into or enables deposition of a new material in the desired pattern upon, the material underneath the photo resist. For example, in complex integrated circuits, a modern CMOS wafer will go through the photolithographic cycle up to 50 times.

Embodiments described herein relate to a microfluidic device and system for measuring vascular occlusion and/or cell adhesion, and particularly relates to a microfluidic device and system for microvascular occlusion rate associated with red blood cell (RBC) deformability and adhesion rate associated with RBC deformability and adhesion. The microfluidic device described herein can mimic architectural features associated with capillary beds of a subject. For example, microfluidic device can mimic 20-μm to 4-μm narrow blood vessels to replicate the specific deformation of RBCs when traversing the microvascular bed through capillaries and small venules. Moreover, the microfluidic device described herein can enable repeated mechanical deformation cycles of RBCs in a wide range to mimic RBCs passing through numerous capillaries during their lifetime. Additionally, to test clinical samples with near-physiological hematocrit levels, the microfluidic device can have wide openings mimicking the arteriovenous anastomoses, which enable full utilization of microvasculature features and prevent complete flow obstruction.

In some embodiments, the microfluidic device can include a plurality of micropillar arrays within a microchannel that define a plurality of microcapillaries that mimic capillary networks of microvasculature of a subject and integrated electrical impedance measurement electrodes to assess RBC-mediated occlusion within the plurality of microcapillaries defined by the micropillar arrays. These microcapillaries can be engineered to retain RBCs with impaired deformability, such that more abnormal RBCs will occlude wide upstream microcapillaries, while those with moderate impairment will occlude finer downstream microcapillaries within the microchannel. The microchannel can also be designed with two wide side or outer openings or passages to mimic arteriovenous anastomoses, which act as shunts in the capillary bed in vivo. These side anastomoses can prevent complete blockade of flow in the microchannel, and enable testing of clinical blood samples with near-physiological hematocrit levels.

Advantageously, RBCs perfused through the microfluidic device experience a wide spectrum of deformations when crossing microcapillaries with different sizes, which recapitulates a more physiologically relevant microenvironment. Additionally, the microfluidic device can examine large numbers of heterogeneous RBCs since the embedded micropillar arrays recapitulate large numbers of microcapillaries with various dimensions, enabling the simultaneous deformability analysis of bulk RBCs at a single-cell level. Moreover, the integrated electrical impedance measurement provides a reproducible functional measurement for standardized assessment of RBC-mediated microvascular occlusion associated with abnormal RBC deformability and an in vitro therapeutic efficacy benchmark for assessing clinical outcome of emerging RBC modifying targeted and curative therapies. The microfluidic device provides a finer and more rapid detection compared to previous devices and an alternative functional metric for standardized measurements of RBC-mediated microvascular occlusion without need for imaging. Furthermore, the microfluidic device can be used to assess very small changes in RBC deformability, under both normoxic (ambient air) and hypoxic conditions, to assess pathologically impaired RBCs in blood. The assessment of pathologically impaired RBCs can be used to assess microvascular health and function of a subject and determine the subject's increased risk of vaso-occlusive crises (VOC) in a range of microcirculatory diseases.

In some examples, the microfluidic device or system can measure or determine RBC-mediated microvascular occlusion and/or adherence of RBCs of a subject. This can be used, for example, to monitor disease severity, treatment response, or treatment effectiveness in a clinically meaningful way.

In one example, the RBCs can be provided in whole blood or can be derived from whole blood of a subject, and the microfluidic system can be used to identify and/or measure the efficacy of therapeutic agents in treating various disorders by measuring the deformability, adherence, and/or occlusion properties of the cells under physiological flow or physiological relevant shear stress conditions and normoxia or hypoxia conditions.

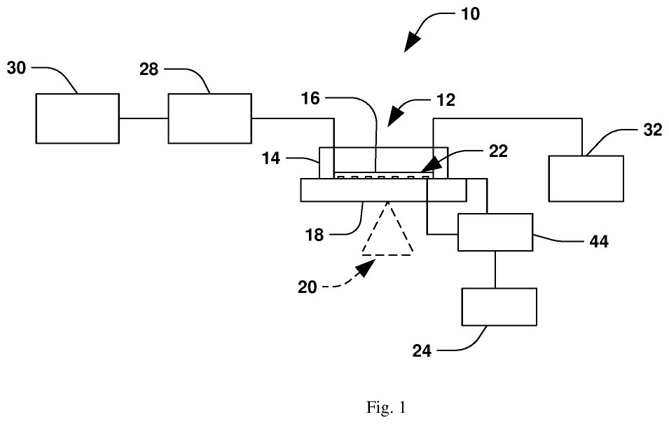

FIG. 1 illustrates a schematic view of a microfluidic system 10 in accordance with an embodiment described herein. The microfluidic system 10 includes a microfluidic device or occlusionchip 12 that has a housing 14 and at least one microchannel 16 in the housing 14 that permits fluid sample flow through the housing 14 along a length of the microchannel 16 from a first end to a second end of the microchannel 16. The microchannel 16 includes at least one cell occlusion region 22 within the microchannel 16. The fluidics associated the microchannels 16 can be arranged such that flow through each microchannel(s) travels in the same direction, or in opposite directions. When a microfluidic device 12 contains at least two microchannels and the fluidics associated the channels are arranged such that flow through each microchannel(s) travels in the same direction, the microchannels are typically either partially fluidically isolated or fluidically isolated. Microchannels that are “fluidically isolated” are configured and designed such that there is no fluid exchanged directly between the microchannels. Microchannels that are “partially fluidically isolated” are configured and designed such that there is partial (e.g., incidental) fluid exchanged directly between the channels.

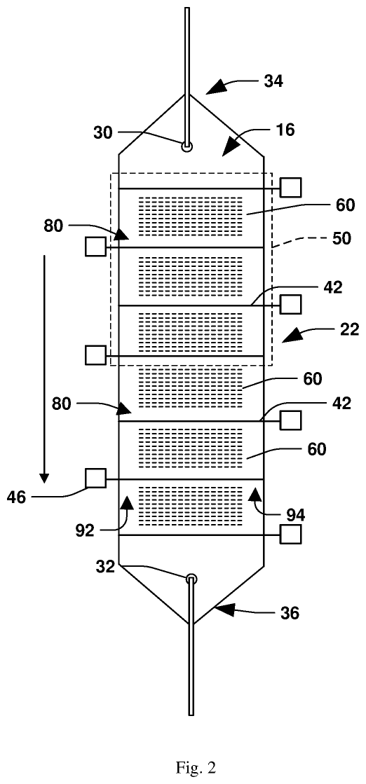

Referring to FIG. 2. the microchannel 16 is fluidly connected to an inlet port 30 at a first end 34 and an outlet port 32 at a second end 36. The inlet port 30 allows fluid to flow through the microchannel 16 from the first end 34 to the second end 36 and out the outlet port 32. The fluid direction is indicated by the arrow. Although FIG. 2 depicts one microchannel, the microfluidic device 12 can include more microchannels.

Referring again to FIG. 1, the housing 14 including the at least one microchannel 16 can optionally contain a substantially planar transparent wall 18 that defines a surface of at least one of the microchannels 16. This substantially planar transparent wall 18, which can be, for example, glass or plastic, permits observation into the microchannel 16 by an optional imaging system 20 (e.g., microscopy) so that at an optical measurement of each cell that passes through the cell occlusion region 22 of one of the microfluidic channels 16 can be obtained. In one example, the transparent wall 18 has a thickness of 0.05 mm to 1 mm. In some cases, the transparent wall 18 may be a microscope cover slip, or similar component. Microscope coverslips are widely available in several standard thicknesses that are identified by numbers, as follows: No. 0-0.085 to 0.13 mm thick, No. 1-0.13 to 0.16 mm thick, No. 1.5-0.16 to 0.19 mm thick, No. 2-0.19 to 0.23 mm thick, No. 3-0.25 to 0.35 mm thick, No. 4-0.43 to 0.64 mm thick, any one of which may be used as a transparent wall 18, depending on the device, microscope, and detection strategy.

In some embodiments, the microchannel(s) 16 may have a depth or height in a range of 0.5 μm to 100 μm, 0.1 μm to 100 μm, 1 μm to 50 μm, 1 μm to 50 μm, 10 μm to 40 μm, 5 μm to 15 μm, 0.1 μm to 5 μm, or 2 μm to 5 μm. The microchannel(s) may have a depth or height of up to 0.5 μm, 1 μm, 1.5 μm, 2.0 μm, 2.5 μm, 3.0 μm, 3.5 μm, 4.0 μm, 4.5 μm, 5.0 μm, 5.5 μm, 6.0 μm, 6.5 μm, 7.0 μm, 7.5 μm, 8.0 μm, 8.5 μm, 9.0 μm, 9.5 μm, 10 μm, 20 μm, 30 μm, 40 μm, 50 μm, 75 μm, 100 μm, or more.

In some embodiments, the at least one microchannel 16 can have a cross-sectional area, perpendicular to the flow direction, of 500 μm², 600 μm², 700 μm², 800 μm², 900 μm², 1000 μm², 2000 μm², 3000 μm², 4000 μm², 5000 μm², 6000 μm², 7000 μm², 8000 μm², 9000 μm², 10,000 μm² or more.

Referring to FIG. 2, the occlusion region 22 of the microchannel 16 can include a plurality of micropillar arrays 40 provided along the length of the microchannel 16 and pairs of uniformly spaced electrodes 42 positioned on opposite sides of each micropillar array 40 in a direction of fluid flow through the microchannel. The micropillar arrays 40 and electrodes 42 can be arranged in series such that a flow path through one micropillar array 40 and over flanking electrodes 42 is parallel with a flow path through the other micropillar arrays 40 and over other flanking electrodes 42 in series. The pairs of electrodes 42 can be configured to measure the electrical impedance of each micropillar array 40.

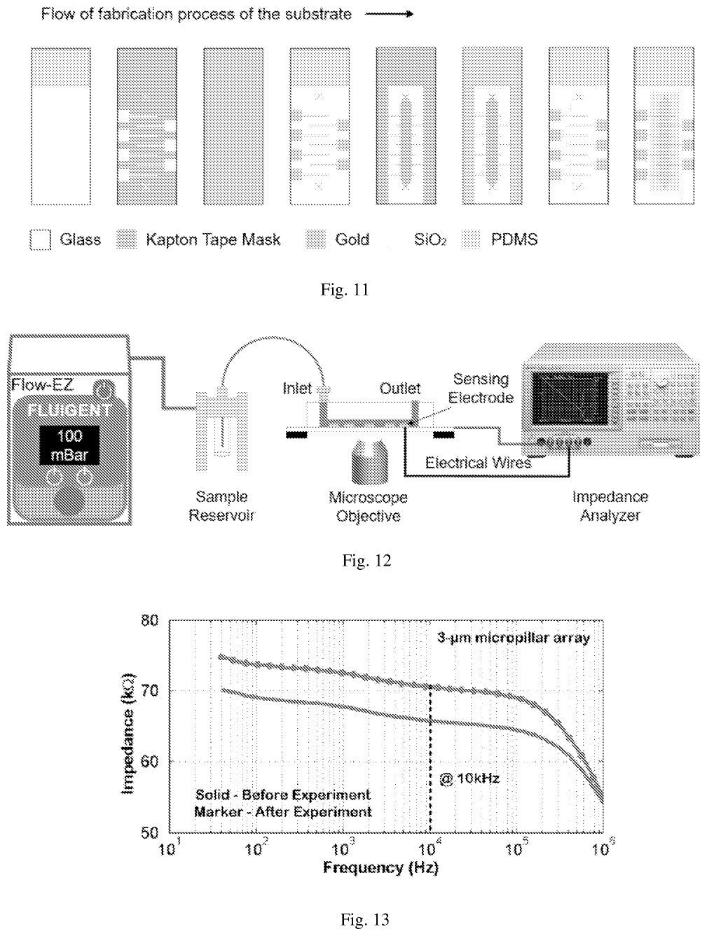

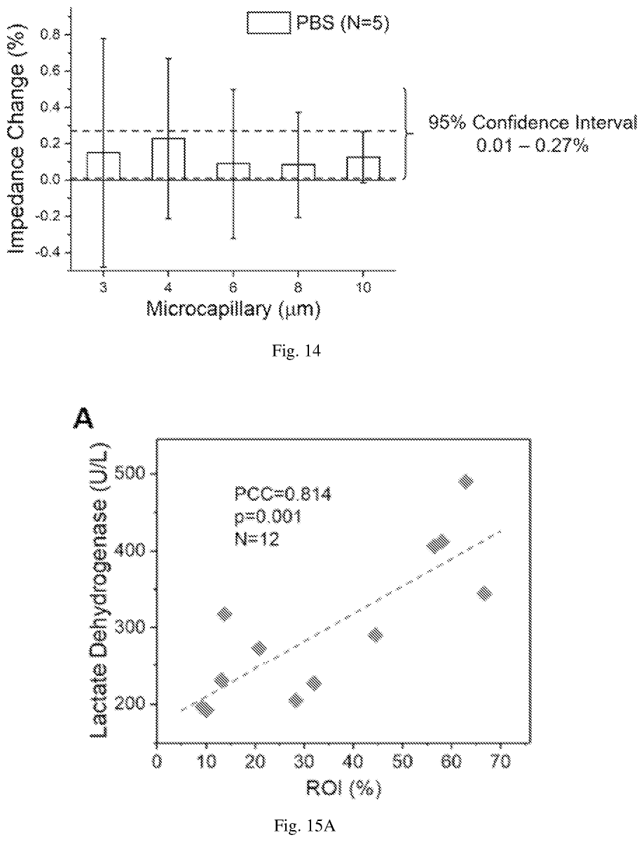

The uniformly spaced pairs of electrodes 42 can be substantially planar and formed on a surface of microchannel 16 through an additive process. The additive process can include but is not limited to depositing, sputtering, printing, coating, or spraying. By way of example as illustrated in FIG. 11, gold electrodes with dimensions of 10.5 mm×0.5 mm with a uniform spacing of 2.5 can be sputter deposited under a Kapton tape mask on a standard microscopic glass slide after which SiO₂ can be deposited under a secondary Kapton mask.

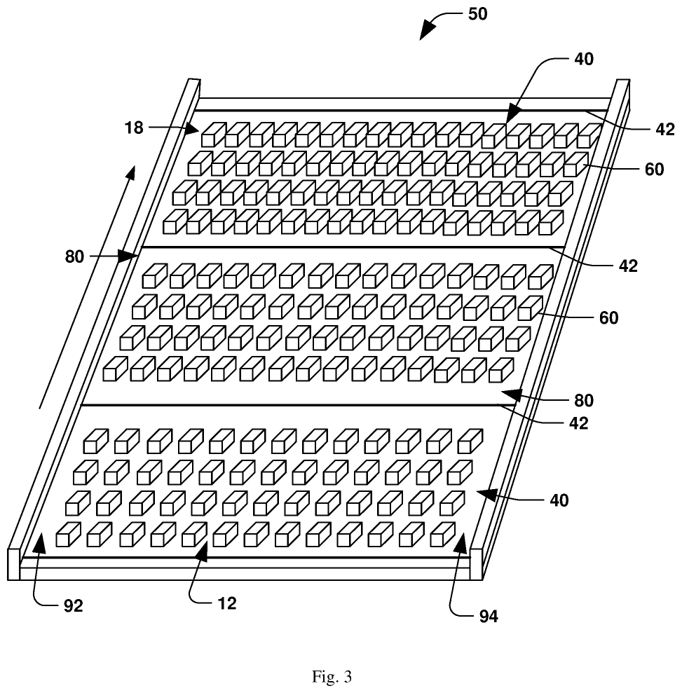

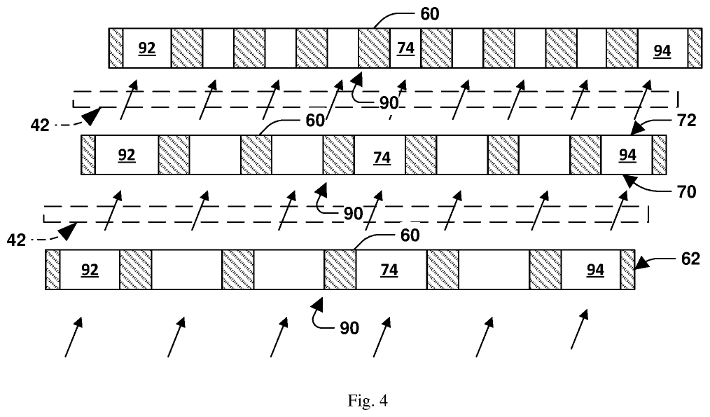

FIG. 3 is a schematic illustration of a cut-out section 50 of the microchannel 16 of FIG. 2. The cut-out section 50 illustrates three micropillar arrays 40 provided in the occlusion region 22 of the microchannel 16. Each micropillar array 40 includes a plurality of micropillars 60 arranged in substantially parallel rows that extend perpendicular to the direction of fluid flow, which is illustrated by the arrow. Pairs of uniformly spaced electrodes 42 configured to measure electrical impedance in each respective micropillar array 40 flank or are positioned on opposite sides of each micropillar array 40. The pairs of electrodes 42 can extend substantially parallel to the rows and perpendicular to the direction of fluid flow. While FIG. 3 shows the micropillars 60 have a shape that is substantially rectangular or box like, it will be appreciated the micropillars 60 of the micropillar arrays 40 can have any of variety of shapes, including, for example, polygonal (e.g., triangular, hexagonal), curvilinear or circular shapes.

In some embodiments, the micropillars 60 of each micropillar array 40 and optionally all the micropillars 60 of the micropillar arrays 40 can have the same shape and size. For example, the micropillars 60 of each micropillar array 40 can have a substantially rectangular cross section and box like shape. In some embodiments, each of the micropillars 60 of the micropillar array 40 can have a width (w), which is parallel to the width of the microchannel, of up to about 1 μm, 2 μm, 3 μm, 4 μm, 5 μm, 6 μm, 7 μm, 8 μm, 9 μm, 10 μm, 11 μm, 12 μm, 13 μm, 14 μm, 15 μm, 16 μm, 17 μm, 18 μm, 19 μm, 20 μm or more, a height (h), which is parallel to the height of the microchannel, of up to about 1 μm, 2 μm, 3 μm, 4 μm, 5 μm, 6 μm, 7 μm, 8 μm, 9 μm, 10 μm, 11 μm, 12 μm, 13 μm, 14 μm, 15 μm, 16 μm, 17 μm, 18 μm, 19 μm, 20 μm or more, and a length, which is parallel to the length of the microchannel, of up to about 5 μm, 6 μm, 7 μm, 8 μm, 9 μm, 10 μm, 11 μm, 12 μm, 13 μm, 14 μm, 15 μm, 16 μm, 17 μm, 18 μm, 19 μm, or 20 μm, 21 μm, 22 μm, 23 μm, 24 μm, 25 μm, 26 μm, 27 μm, 28 μm, 29 μm, or 30 μm or more. In one example, each micropillar 60 of the micropillar array 40 can have a width of about 10 μm, a height of about 12 μm, and a length of about 20 μm.

The micropillars 60 of each micropillar array 40 can be arranged in evenly or uniformly spaced rows 62. The rows 62 of micropillars 60 can extend perpendicular to fluid flow through the microchannel 16. The rows 62 of micropillars 60 in each micropillar array 40 can be substantially parallel to one another. The number of rows 62 of micropillars 60 in each micropillar array 40 can be the same or vary.

In some embodiments, each micropillar array can include at least 3, 4, 5, 6, 7, 8, 9, 10, or more rows of micropillars 60. In one example, each micropillar array 40 can include about 5 to about 10 rows that are even spaced from each other by, for example, about 5 μm, 6 μm, 7 μm, 8μ, 9 μm, 10μ, 11μ, 12μ, 13 μm, 14μ, 15μ, 16 μm, 17 μm, 18 μm, 19 μm, or 20 μm, 21 μm, 22 μm, 23 μm, 24 μm, 25 μm, 26 μm, 27 μm, 28 μm, 29 μm, or 30 μm or more.

The number of micropillars 60 in each row 62 can vary depending on the width of the microchannel 16, the width of the micropillars 60, and the spacing between respective micropillars 60. In some embodiments, at least 10, 11, 12, 13, 14, 15, 16, 17, 18, 19, 20, 21, 22, 23, 24, 25, 26, 27, 28, 29, 30, 31, 32, 33, 34, 35, 36, 27, 38, 39, 40, 41, 42, 43, 44, 45, 46, 47, 48, 49, 50 or more uniformly spaced micropillars 60 can be arranged in row of each micropillar array 40.

The micropillars 60 in each row 62 can be evenly or uniformly spaced from each other such that the distance between each micropillar 60 in a respective row 62 of each micropillar array 40 is substantially the same. The distance between each micropillar in a respective row will vary depending upon the micropillar array and can be, for example, about 1 μm, 2 μm, 3 μm, 4 μm, 5 μm, 6 μm, 7 μm, 8 μm, 9 μm, 10 μm, 11 μm, 12 μm, 13 μm, 14 μm, 15 μm, 16 μm, 17 μm, 18 μm, 19 μm, 20 μm or more.

Successive micropillar arrays 40 in path of fluid flow through the microchannel 16 have decreasing separation distances between the micropillars 60 in the rows 62. For example, micropillars in a first micropillar array at the first end of the microchannel can be separated from each other in a row by about 12 μm, micropillars in a successive second micropillar array downstream of the first micropillar array can be separated from each other in a row by about 10 μm, micropillars in a successive third micropillar array downstream of the second micropillar array can be separated from each other in a row by about 8 μm, micropillars in a successive fourth micropillar array downstream of the third micropillar array can be separated from each other in a row by about 6 μm, micropillars in a successive fifth micropillar array downstream of the fourth micropillar array can be separated from each other in a row by about 4 μm, and micropillars in a successive sixth micropillar array downstream of the fifth micropillar array can be separated from each other in a row by about 3 μm.

Referring to FIG. 4 which is a schematic illustration of cross-section of flow through a single row of micropillars of successive micropillar arrays, the micropillars 60 of the micropillar arrays 40 can extend from a substantially planar lower surface 70 to a substantially planar upper surface 74 of the microchannel 16 such that the micropillars 60 of each row of the micropillar array 40 define a plurality of microcapillaries 74 having a separation that is defined by the width and height of the micropillars in a respective row of a micropillar array. The plurality of microcapillaries 74 defined by each micropillar array 40 can be arranged in series with the electrodes 42 such that a flow path through one microcapillary 74 and over one electrode 42 is parallel with a flow path through the other microcapillaries 74 and over other electrodes 42. The microcapillaries 74 defined by the micropillars in each row 62 can have substantially the same width, height, and cross-sectional area.

Referring to FIGS. 3 and 4, the separation distance of the microcapillaries 74 defined by each micropillar array 40 decreases in a direction of fluid flow through the microchannel 16 in accordance with decreasing distance between each micropillar 74 in each micropillar array 40. The decreasing separation distance of the microcapillaries 74 of the successive micropillar arrays 40 can mimic capillary networks of a subject and be engineered to retain cells, such RBCs, with impaired deformability so that more abnormal RBCs will occlude wide upstream microcapillaries 74, while those with moderate impairment will occlude finer downstream microcapillaries 74 within the microchannel 16.

Each of the microcapillaries 74 defined by a respective micropillar array 40 can have a substantially uniform width, height, and cross-sectional area perpendicular to the flow direction through the microcapillaries 74. The micropillars 60 in successive rows 62 of each micropillar array 40 can be aligned directly behind or offset from the micropillars 60 in a preceding row 62 such that microcapillaries 74 defined by a successive or preceding row 62 are offset perpendicular to the direction of fluid flow.

In some embodiments, a micropillar array 40 at the first end 34 defines a plurality of microcapillaries that can each have a separation distance or width of about 12 μm. Each successive micropillar array in the direction of fluid flow through the microchannel can define a plurality of microcapillaries that can each have a separation distance of at least about 1%, about 5%, about 10%, about 15%, about 20%, about 25%, about 30%, about 35%, about 40%, about 45%, about 50%, about 55%, or about 60% less (e.g., about 10% to about 30% less) than a plurality of microcapillaries defined by a preceding micropillar array. For example, microcapillaries defined by a successive second micropillar arrays downstream of the micropillar array at first end can each have a separation distance of about 11 μm, about 10 μm, about 9 μm, about 8 μm, about 7 μm, about 6 μm, about 5 μm, about 4 μm, about 3 μm or less.

In some embodiments, a micropillar array 40 at the first end 34 defines a plurality of microcapillaries that can each have a cross sectional area of about 200 μm² to about 250 μm² (e.g., about 230 μm² to about 250 μm²). Each successive micropillar array in the direction of fluid flow through the microchannel can define a plurality of microcapillaries that can each have a cross sectional area at least about 1%, about 5%, about 10%, about 15%, about 20%, about 25%, about 30%, about 35%, about 40%, about 45%, about 50%, about 55%, or about 60% less (e.g., about 10% to about 30% less) than a plurality of microcapillaries defined by a preceding micropillar array. For example, microcapillaries defined by a successive second micropillar arrays downstream of the micropillar array at first end can each have successive cross sectional areas of about 220 μm² to about 230 μm², about 210 μm² to about 220 μm², about 200 μm² to about 210 μm², about 190 μm² to about 200 μm², about 180 μm² to about 190 μm², about 170 μm² to about 190 μm², about 160 μm² to about 170 μm², about 150 μm² to about 160 μm², about 140 μm² to about 150 μm², about 130 μm² to about 140 μm², about 120 μm² to about 130 μm², about 110 μm² to about 120 μm², about 100 μm² to about 110 μm², about 90 μm² to about 100 μm², about 80 μm² to about 90 μm², about 70 μm² to about 80 μm², about 60 μm² to about 70 μm², about 50 μm² to about 60 μm², about 40 μm² to about 50 μm², or about 30 μm² to about 40 μm²

In other embodiments, a micropillar array 40 at the second end 36 defines a plurality of microcapillaries that can each have a separation distance or width of about 3 μm. Each preceding micropillar array opposite the direction of fluid flow through the microchannel from the second end 36 can define a plurality of microcapillaries that can each have a separation distance at least about 1%, about 5%, about 10%, about 15%, about 20%, about 25%, about 30%, about 35%, about 40%, about 45%, about 50%, about 55%, or about 60% greater (e.g., about 20% to about 50% greater) than a plurality of microcapillaries defined by a preceding micropillar array. For example, microcapillaries defined by a preceding micropillar array upstream of the micropillar array at the second end can each have a width of about 4 μm, about 5 μm, about 6 μm, about 7 μm, about 8 μm, about 9 μm, about 10 μm, about 11 μm, about 12 μm, or more.

In other embodiments, the microchannel 16 includes a micropillar array 40 at the second end 36 that defines a plurality of microcapillaries that can each have a cross sectional area of about 40 μm² to about 50 μm². Each preceding micropillar array opposite the direction of fluid flow through the microchannel from the second end 36 can define a plurality of microcapillaries that can each have a cross sectional area at least about 1%, about 5%, about 10%, about 15%, about 20%, about 25%, about 30%, about 35%, about 40%, about 45%, about 50%, about 55%, or about 60% greater (e.g., about 5% to about 50% greater) than a plurality of microcapillaries defined by a preceding micropillar array. For example, microcapillaries defined by a preceding micropillar array upstream of the micropillar array at the second end can each have a cross sectional area of about 50 μm² to about 60 μm², about 60 μm² to about 70 μm², about 70 μm² to about 80 μm², about 80 μm² to about 90 μm², about 90 μm² to about 100 μm², about 100 μm² to about 110 μm², about 110 μm² to about 120 μm², about 120 μm² to about 130 μm², about 130 μm² to about 140 μm², about 140 μm² to about 150 μm², about 160 μm² to about 170 μm², about 170 μm² to about 180 μm², about 180 μm² to about 190 μm², about 190 μm² to about 200 μm², about 200 μm² to about 210 μm², about 210 μm² to about 220 μm², about 220 μm² to about 230 μm², about 230 μm² to about 240 μm², or about 240 μm² to about 250 μm².

In some embodiments, the separation distance of the microcapillaries 74 defined by at least one of the plurality of micropillar arrays 40 permits passage of healthy cells in a fluid sample perfused through the microchannel but is occluded by cells with impaired deformability, such as abnormal RBCs. The cell can be blood cells, such as RBCs.

In other embodiments, the separation distance of the plurality of microcapillaries 74 at the second end 36 of the microchannel 16 can be such that the microcapillaries are occluded cells in a fluid sample perfused through the at least one microchannel 16.

In some embodiments, as illustrated in FIGS. 2 and 3, successive micropillar arrays 40 can be separated from each other in the microchannel by a gap region 80, which is free of micropillars 60 but that includes one of the electrodes 42. In one example, this gap region 80 is of a length that allows cells, such as RBCs, in the fluid sample to recover, at least partially, their shape after passing through the microcapillaries of a respective micropillar array. In another example, the gap region is of a length that does not allow one or more cells in the fluid sample to recover its shape after passing through the microcapillaries of a respective micropillar array.

The gap region 80 may have a length (e.g., distance between respective micropillar arrays) of up to 20 μm, 21 μm, 22 μm, 23 μm, 24 μm, 25 μm, 26 μm, 27 μm, 28 μm, 29 μm, 30 μm, 31 μm, 32 μm, 33 μm, 34 μm, 35 μm, 36 μm, 37 μm, 38 μm, 39 μm, 40 μm, 41 μm, 42 μm, 43 μm, 44 μm, 45 μm, 46 μm, 47 μm, 48 μm, 49 μm, 50 μm, 55 μm, 60 μm, 65 μm, 70 μm, 75 μm, 80 μm, 85 μm, 90 μm, 95 μm, 100 μm, 150 μm, 200 μm, 250 μm, 300 μm, 350 μm, 400 μm, 450 μm, 500 μm, 1 mm or more. For example, the gap region may have a length in a range of 25 μm to 100 μm, 50 μm to 125 μm, 75 μm to 150 μm, or 100 μm to 200 μm.

In some embodiments, each of the micropillar arrays 40 can be arranged in an inner portion 90 of the microchannel 16 that extends the length of the microchannel 16. The microchannel 16 can include two parallel outer or side passages 92 and 94 on opposite sides of the inner portion 90 that extend the length of the microchannel 16. The outer passages 92 and 94 are designed to mimic arteriovenous anastomoses which act as shunts in the capillary bed in vivo. These outer passages 92 and 94 can prevent complete blockade of flow in the microchannel, and enable testing of clinical blood samples with near-physiological hematocrit levels. The outer passages 92 and 94 can be in fluid communication with the plurality of microcapillaries 74 defined by the plurality micropillar arrays 40. The outer passages can have cross sectional areas or separation distances that permit cells in a fluid sample to flow through the microchannel without being occluded and/or obstructed. In some embodiment the outer passages 92 and 94 can have widths of 30 μm, 31 μm, 32 μm, 33 μm, 34 μm, 35 μm, 36 μm, 37 μm, 38 μm, 39 μm, 40 μm, 41 μm, 42 μm, 43 μm, 44 μm, 45 μm, 46 μm, 47 μm, 48 μm, 49 μm, 50 μm, 55 μm, 60 μm, 65 μm, 70 μm, 75 μm, 80 μm, 85 μm, 90 μm, 95 μm, 100 μm, 150 μm, 200 μm, 250 μm, 300 μm, 350 μm, 400 μm, 450 μm, 500 μm, 1 mm or more. For example, the outer passages 92 and 94 may have widths in a range of 25 μm to 100 μm, 50 μm to 125 μm, 75 μm to 150 μm, or 100 μm to 200 μm

Optionally, where the microfluidic device 12 is designed to occlude all cells that pass through the microchannel, the outer passages can be omitted so that the micropillar arrays 40 extend to opposite walls that define the width of the microchannel. In this instance, fluid passing through the microchannel will not be able to bypass the microcapillaries defined by the micropillar arrays.

In some embodiments, the microfluidic system 10 can simulate physiologically relevant shear gradients (e.g., 0.5 dynes/cm² to about 2 dynes/cm²) of microcirculatory blood flow at a constant single volumetric flow rate. Using this system, shear-dependent adhesion, occlusion, and deformability of cells, for example, RBCs and WBCs, from subjects with disorders, such as SCD can be investigated to determine complex dynamic interactions between RBC-mediated microcirculatory occlusion and clinical outcomes in SCD. These interactions may also be relevant to other microcirculatory disorders

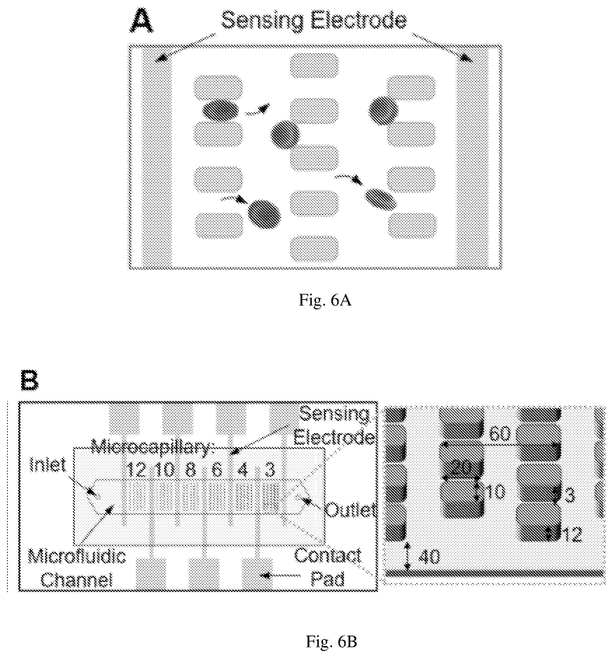

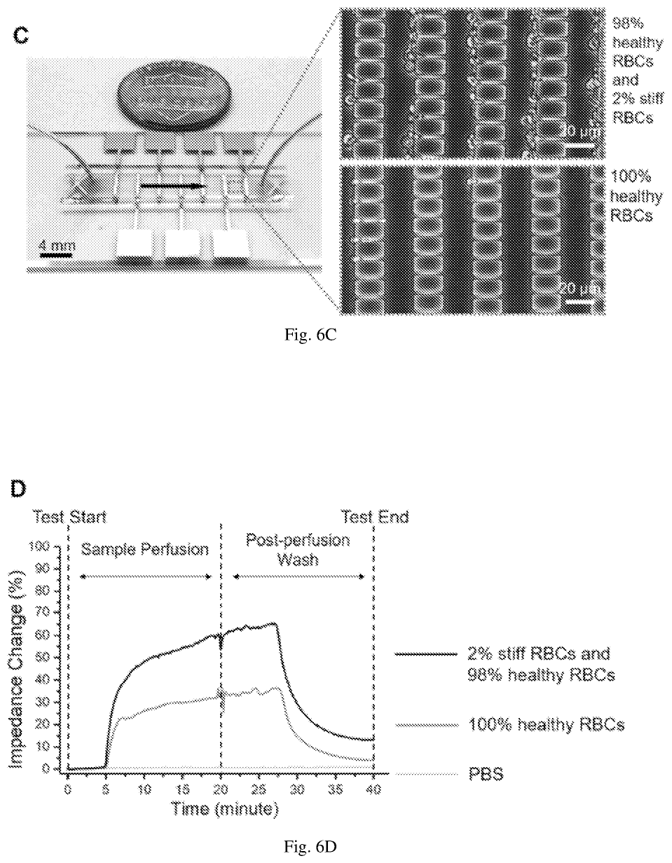

In some embodiments, as illustrated in FIG. 6C and FIG. 11 the microfluidic device can include a multilayer structure formed of a base layer, a microchannel, intermediate layers, and a cover layer. The microchannel includes the occlusion region. Referring to FIG. 2, a first end 34 of the microchannel 16 is aligned with a corresponding inlet port 30. A second end 36 of the microchannel 16 is aligned with a corresponding outlet port 32. This creates a flow channel from an inlet port 30 to the corresponding outlet 32 port via the microchannel 16. The microchannel 16 can also extend slightly beyond its respective inlet port 30 and outlet port 32. The microchannel is sized to accept volumes, e.g., μl or mL, of the fluid sample containing cells that occlude and/or adhered or captured in the occlusion region.

Referring again to FIG. 6C, the base layer provides structural support to the microchannel and is formed of a sufficiently rigid, optically transparent, and gas impermeable material, such as poly(methyl methacrylate) (PMMA) or glass. The base layer can have a suitable thickness, for example of about 0.1 mm to about 2 mm, or about 1.6 mm, determined by manufacturing and assembly restrictions and include electrodes that are provided thereon by, for example, sputtering.

The cover layer contains the inlet ports and outlet ports used to feed the sample in/out of the microchannel. The cover layer thickness can be about 1 mm to about 10 mm, for example, about 3.6 mm, and is determined by the integration and assembly requirements. The inlet and outlet port diameters can be about 0.3 mm to about 3 mm, for example about 1 mm. The lower size limit is determined by the manufacturing restrictions. The upper size limit is determined by the desired flow conditions of sample through the channel. In another example (not shown), a laser cutter can be used to cut a larger piece of PMMA into a desired size for the microfluidic device and to cut holes for the inlet ports and the outlet ports.

The microchannel can include a plurality of micropillar arrays that can be fabricated through photolithography. By way of example, a photomask with designed microchannel features can be used to pattern a silicon wafer. The wafer can be spin-coated with a negative photoresist layer, soft-baked, and exposed to UV light under the photomask. After post-exposure baking, developing and hard baking, the master wafer is completed. The wafer can be covered with PDMS and then cured. The cured PDMS block can then peeled-off from the master wafer, and two holes punched as the inlet and the outlet to define the microchannel. After cleaning, the PDMS block, which forms the microchannel and micropillar arrays, can be bonded to the base layer.

The intermediate layers can be adhered to the base layer around the microchannel after the microchannel is placed on the base layer. The cover layer, which can have the same lateral dimensions as the base layer and the intermediate layer, can be adhered onto the exposed side of the intermediate layer, thereby enclosing the microchannel. In the examples depicted, the microfluidic device is oriented such that the cover layer is on top. Alternatively, the microfluidic device can be oriented such that the cover layer is on the bottom (not shown).

In some embodiments, at least one surface of the cell occlusion region 22 of the microchannel 16, including the surface of the micropillars, can be functionalized with at least one capturing agent or bioaffinity ligand that captures or adheres a cell of interest to a surface of the microchannel when a sample fluid containing cells is passed or perfused through the at least one microchannel. If the housing includes multiple microchannels, each microchannel can be functionalized with a different capturing agent to adhere different cells of interest thereto. In any case, each microchannel is configured to receive and provide cell adhesion analysis of a microvolume fluid sample.

In some embodiments, the capturing agents can include, for example, bioaffinity ligands or adhesion molecules that are associated with an activated phenotype in a hematological or circulatory disease or disorder, such as SCD. Such bioaffinity ligands or adhesion molecules can include, for example, at least one of laminin, fibronectin, selectins, such as E-Selectin, P-Selectin, or L-selectin, intracellular adhesion molecule 1 (ICAM-1), or vascular cellular adhesion molecule 1 (VCAM-1). Laminin, fibronectin, E-Selectin, P-Selectin, L-selectin, ICAM-1, or VCAM-1 can adhere to cells, such as WBCs and/or RBCs, and be used to detect and/or measure WBC and/or RBC adherence under physiological relevant shear stress and normoxic and hypoxic conditions.

The capturing agent or bioaffinity ligand can be adhered to, functionalized or chemically functionalized to the at least one surface of the cell occlusion region of the microchannel. The bioaffinity ligands may be functionalized to the at least one surface of the cell occlusion region covalently or non-covalently. A linker can be used to provide covalent attachment of a bioaffinity ligand to the surface of the cell occlusion region. The linker can be a linker that can be used to link a variety of entities.

In some examples, the linker may be a homo-bifunctional linker or a hetero-bifunctional linker, depending upon the nature of the molecules to be conjugated. Homo-bifunctional linkers have two identical reactive groups. Hetero-bifunctional linkers have two different reactive groups. Various types of commercially available linkers are reactive with one or more of the following groups: primary amines, secondary amines, sulphydryls, carboxyls, carbonyls and carbohydrates. Examples of amine-specific linkers are bis(sulfosuccinimidyl) suberate, bis[2-(succinimidooxycarbonyloxy)ethyl]sulfone, disuccinimidyl suberate, disuccinimidyl tartarate, dimethyl adipimate 2HCl, dimethyl pimelimidate 2HCl, dimethyl suberimidate HCl, ethylene glycolbis-[succinimidyl-[succinate]], dithiolbis(succinimidyl propionate), and 3,3′-dithiobis(sulfosuccinimidylpropionate). Linkers reactive with sulfhydryl groups include bismaleimidohexane, 1,4-di-[3′-(2′-pyridyldithio)-propionamido)]butane, 1-[p-azidosalicylamido]-4-[iodoacetamido]butane, and N-[4-(p-azidosalicylamido)butyl]-3′-[2′-pyridyldithio]propionamide. Linkers preferentially reactive with carbohydrates include azidobenzoyl hydrazine. Linkers preferentially reactive with carboxyl groups include 4-[p-azidosalicylamido]butylamine.

Heterobifunctional linkers that react with amines and sulfhydryls include N-succinimidyl-3-[2-pyridyldithio]propionate, succinimidyl[4-iodoacetyl]aminobenzoate, succinimidyl 4-[N-maleimidomethyl]cyclohexane-1-carboxylate, m-maleimidobenzoyl-N-hydroxysuccinimide ester, sulfosuccinimidyl 6-[3-[2-pyridyldithio]propionamido]hexanoate, and sulfosuccinimidyl 4-[N-maleimidomethyl]cyclohexane-1-carboxylate. Heterobifunctional linkers that react with carboxyl and amine groups include 1-ethyl-3-[3-dimethylaminopropyl]-carbodiimide hydrochloride. Heterobifunctional linkers that react with carbohydrates and sulfhydryls include 4-[N-maleimidomethyl]-cyclohexane-1-carboxylhydrazide HCl, 4-(4-N-maleimidophenyl)-butyric acid hydrazide.2HCl, and 3-[2-pyridyldithio]propionyl hydrazide.

In some embodiments, a surface layer of 3-aminopropyl triethoxy silane (APTES) and/or (3-mercaptopropyl) trimethoxysilane (MTPMS) can be initially applied to surfaces of the microchannel followed by incubation with N-γ-maleimidobutyryl-oxysuccinimide ester (GMBS) to functionalize the bioaffinity ligand or capturing agent to the surfaces.

By way of example, a GMBS working solution can prepared by dissolving GMBS in DMSO and diluting with ethanol. A bioaffinity ligand described herein, such as laminin, fibronectin, E-Selectin, P-Selectin, L-selectin, ICAM-1, or VCAM-1 can be diluted with PBS to create a bioaffinity ligand working solution. The GMBS working solution can injected into the microchannels and incubated at room temperature. Following GMBS incubation, the microchannels can be washed. Next, the bioaffinity ligand working solution can injected into the microchannels and incubated at room temperature. The surface can then passivated by injecting a BSA solution incubated, thereby forming a bioaffinity ligand functionalized surface. The microchannels can be optionally rinsed with PBS before processing samples.

Alternatively, the bioaffinity ligands may be non-covalently coated onto a surface of the cell occlusion region. Non-covalent deposition of the bioaffinity ligand to the surface of the cell occlusion region may involve the use of a polymer matrix. The polymer may be naturally occurring or non-naturally occurring and may be of any type including but not limited to nucleic acid, e.g., DNA, RNA, PNA, LNA, and the like or mimics, derivatives or combinations thereof, amino acid, e.g., peptides, proteins (native or denatured), and the like or mimics, derivatives or combinations thereof, lipids, polysaccharides, and functionalized block copolymers. The bioaffinity ligand may be adsorbed onto and/or entrapped within the polymer matrix. Alternatively, the bioaffinity ligand may be covalently conjugated or crosslinked to the polymer, e.g., it may be “grafted” onto a functionalized polymer.

An example of a suitable peptide polymer is poly-lysine, e.g., poly-L-lysine. Examples of other polymers include block copolymers that comprise polyethylene glycol (PEG), polyamides, polycarbonates, polyalkylenes, polyalkylene glycols, polyalkylene oxides, polyalkylene terepthalates, polyvinyl alcohols, polyvinyl ethers, polyvinyl esters, polyvinyl halides, polyvinylpyrrolidone, polyglycolides, polysiloxanes, polyurethanes, alkyl cellulose, hydroxyalkyl celluloses, cellulose ethers, cellulose esters, nitrocelluloses, polymers of acrylic and methacrylic esters, methyl cellulose, ethyl cellulose, hydroxypropyl cellulose, hydroxypropyl methyl cellulose, hydroxybutyl methyl cellulose, cellulose acetate, cellulose propionate, cellulose acetate butyrate, cellulose acetate phthalate, carboxylethyl cellulose, cellulose triacetate, cellulose sulphate sodium salt, poly(methyl methacrylate), poly(ethyl methacrylate), poly(butylmethacrylate), poly(isobutyl methacrylate), poly(hexylmethacrylate), poly(isodecyl methacrylate), poly(lauryl methacrylate), poly(phenyl methacrylate), poly(methyl acrylate), poly(isopropyl acrylate), poly(isobutyl acrylate), poly(octadecyl acrylate), polyethylene, polypropylene, poly(ethylene glycol), poly(ethylene oxide), poly(ethylene terephthalate), poly(vinyl alcohols), polyvinyl acetate, polyvinyl chloride, polystyrene, polyhyaluronic acids, casein, gelatin, glutin, polyanhydrides, polyacrylic acid, alginate, chitosan, poly(methyl methacrylates), poly(ethyl methacrylates), poly(butylmethacrylate), poly(isobutyl methacrylate), poly(hexylmethacrylate), poly(isodecyl methacrylate), poly(lauryl methacrylate), poly(phenyl methacrylate), poly(methyl acrylate), poly(isopropyl acrylate), poly(isobutyl acrylate), and poly(octadecyl acrylate), poly(lactide-glycolide), copolyoxalates, polycaprolactones, polyesteramides, polyorthoesters, polyhydroxybutyric acid, polyanhydrides, poly(styrene-b-isobutylene-b-styrene) (SIBS) block copolymer, ethylene vinyl acetate, poly(meth)acrylic acid, polymers of lactic acid and glycolic acid, polyanhydrides, poly(ortho) esters, polyurethanes, poly(butic acid), poly(valeric acid), and poly(lactide-cocaprolactone), and natural polymers such as alginate and other polysaccharides including dextran and cellulose, collagen, albumin and other hydrophilic proteins, zein and other prolamines and hydrophobic proteins, copolymers and mixtures thereof, and chemical derivatives thereof including substitutions and/or additions of chemical groups, for example, alkyl, alkylene, hydroxylations, oxidations, and other modifications routinely made by those skilled in the art.

Referring again to FIG. 1, the microfluidic system 10 can further include a reservoir 28 fluidically connected with the one or more microfluidic channels 16, and a pump 30 that perfuses fluid from the reservoir 28 through the one or more microchannels 16 to a waste or fluid collection reservoir 32. The pump 30 can designed and configured to create a pressure to create a pressure (gauge pressure) in at least one of the microchannels 16 of up to 50 Pa, 100 Pa, 200 Pa, 300 Pa, 400 Pa, 500 Pa, 600 Pa, 700 Pa, 800 Pa, 900 Pa, 1 kPa, 2 kPa, 5 kPa, 10 kPa or more. The pump 30 may be designed and configured to create a pressure (gauge pressure) in the channel in a range of 50 Pa to 200 Pa, 100 Pa to 500 Pa, 100 Pa to 800 Pa, 100 Pa to 1 kPa, 500 Pa to 5 kPa, or 500 Pa to 10 kPa.

The microfluidic system 10 may also be designed and configured to create an average fluid flow velocity within the channel of up to about 100 μm/s, about 200 μm/s, about 500 μm/s, about 1 mm/s, about 2 mm/s, about 5 mm/s, about 10 mm/s, or more.

The microfluidic system 10 may be designed and configured to create an average fluid velocity within at least one microchannel 16 in a range of 1 mm/s to 5 mm/s, 1 mm/s to 10 mm/s, 1 mm/s to 20 mm/s, 1 mm/s to 50 mm/s, 10 μm/s to 10 mm/s, or 100 μm/s to 2 mm/s.

In some embodiments, the fluid sample can flow through the at least one microchannel at a physiologically relevant flow velocity. The physiologically relevant flow velocity can be about 1 mm/s to about 2 mm/s.

In some embodiments, the reservoir 28 contains cells, such as RBCs and WBCs, suspended in a fluid, such as blood or plasma.

The microfluidic system also includes an impedance measuring system 44 that is electrically connected via electrical connectors or leads to contact pads 46 (FIG. 2) of pairs of electrode. The impedance measuring system can be measure RBC pathology or RBC-mediated occlusion of microcapillaries associated with deformability of RBCs in respective micropillar arrays by measuring the impedance of each micropillar array when a fluid sample containing cells, such as RBCs, is passed or perfused through the microchannel under, for example, physiological relevant flow velocity and normoxia or hypoxia conditions.

The impedance measuring system 44 includes control unit 24, which can include a computer readable storage medium and a processor (not shown) configured to compare and/or determine changes of measured impedances of pairs of electrodes 42 and provide real-time feedback to a subject of the results of the impedance measurement. These results, in turn, can be readily transmitted to a primary care provider and/or stored in a medical record database.

The impedance processing may be implemented using hardware, software or a combination thereof. When implemented in software, the software code can be executed on any suitable processor or collection of processors, whether provided in a single computer or distributed among multiple computers. Such processors may be implemented as integrated circuits, with one or more processors in an integrated circuit component. Though, a processor may be implemented using circuitry in any suitable format.

Further, it should be appreciated that a computer may be embodied in any of a number of forms, such as a rack-mounted computer, a desktop computer, a laptop computer, or a tablet computer. Additionally, a computer may be embedded in a device not generally regarded as a computer but with suitable processing capabilities, including a Personal Digital Assistant (PDA), a smart phone or any other suitable portable or fixed electronic device.

Also, a computer may have one or more input and output devices. These devices can be used, among other things, to present a user interface. Examples of output devices that can be used to provide a user interface include printers or display screens for visual presentation of output and speakers or other sound generating devices for audible presentation of output. Examples of input devices that can be used for a user interface include keyboards, and pointing devices, such as mice, touch pads, and digitizing tablets. As another example, a computer may receive input information through speech recognition or in other audible format.

Such computers may be interconnected by one or more networks in any suitable form, including as a local area network or a wide area network, such as an enterprise network or the Internet. Such networks may be based on any suitable technology and may operate according to any suitable protocol and may include wireless networks, wired networks or fiber optic networks

Also, the various methods or processes outlined herein may be coded as software that is executable on one or more processors that employ any one of a variety of operating systems or platforms. Additionally, such software may be written using any of a number of suitable programming languages and/or programming or scripting tools, and also may be compiled as executable machine language code or intermediate code that is executed on a framework or virtual machine.

In this respect, a computer readable medium (or multiple computer readable media) (e.g., a computer memory, one or more floppy discs, compact discs (CD), optical discs, digital video disks (DVD), magnetic tapes, flash memories, circuit configurations in Field Programmable Gate Arrays or other semiconductor devices, or other non-transitory, tangible computer storage medium) can be encoded with one or more programs that, when executed on one or more computers or other processors, perform methods that implement the various embodiments described herein. The computer readable medium or media can be transportable, such that the program or programs stored thereon can be loaded onto one or more different computers or other processors to implement various aspects described herein. As used herein, the term “non-transitory computer-readable storage medium” encompasses only a computer-readable medium that can be considered to be a manufacture (i.e., article of manufacture) or a machine.

The terms “program” or “software” are used herein in a generic sense to refer to any type of computer code or set of computer-executable instructions that can be employed to program a computer or other processor to implement various aspects as discussed above. Additionally, it should be appreciated that according to one aspect of this embodiment, one or more computer programs that when executed perform methods of described herein need not reside on a single computer or processor, but may be distributed in a modular fashion amongst a number of different computers or processors to implement various aspects herein.

Computer-executable instructions may be in many forms, such as program modules, executed by one or more computers or other devices. Generally, program modules include routines, programs, objects, components, data structures, etc. that perform particular tasks or implement particular abstract data types. Typically, the functionality of the program modules may be combined or distributed as desired in various embodiments.

The processor can determine cumulative impedance changes of pairs of electrodes flanking at least one micropillar array by comparing the impedance of the pair of electrodes before and after a fluid sample including RBCs, such as a blood sample, is perfused through the microchannel. The compared impedance changes can be used to determine RBC-mediated occlusion of the microcapillaries of each respective micropillar array and RBC pathology.

In some embodiments, the RBCs are from a subject at risk of a vaso-occlusive crises and/or decreased microvascular health and function. The subject can have an increased risk of vaso-occlusive crises (VOC) and/or decreased microvascular health and function when the REI is greater than a control value.

In some embodiments, the fluid sample includes at least about 1%, at least about 5%, at least about 10%, at least about 15%, at least about 20%, at least about 30%, or at least about 40% hematocrit. The RBCs can be from stored blood and/or blood to be transfused. The fluid sample can also be whole blood obtained from a subject.

In some embodiments, the first impedance and the second impedance are measured at a frequency of about 40 Hz to about 1 MHz, about 100 Hz to about 100 kHz, or about 1 kHz to about 50 kHz.

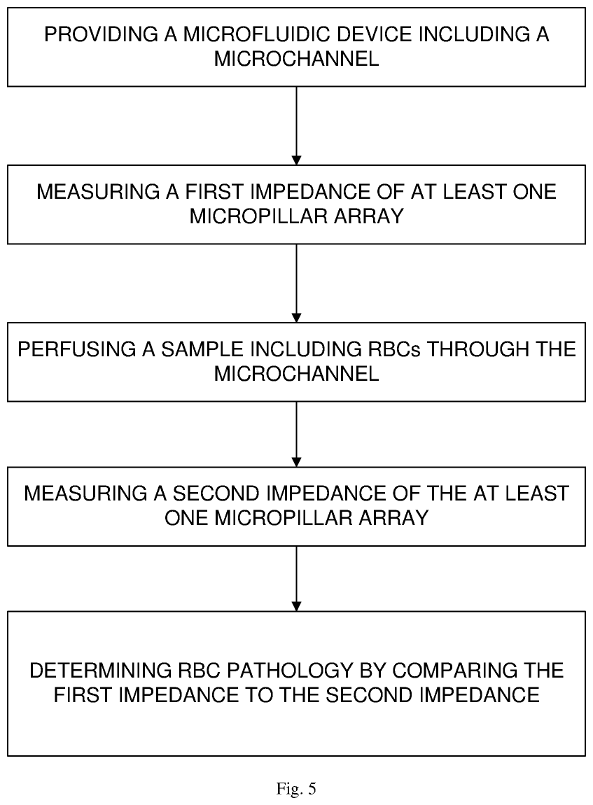

In some embodiments, as illustrated in a flow diagram of FIG. 5, RBC pathology and/or RBC-mediated occlusion can be measured or determined by measuring a first or baseline impedance of at least one micropillar array before the fluid sample including RBCs is perfused through the at least one microchannel. The first impedance is measured while phosphate buffered saline (PBS) is perfused through the microchannel. The fluid sample including the RBCs can then be perfused through the at least one microchannel of the microfluidic device. A second impedance of the at least one micropillar array can be measured after perfusion of the fluid sample through the microchannel. PBS can be perfused through the microchannel after perfusing the fluid sample but before the second impedance measurement. The RBC pathology can then be determined by comparing the first impedance and the second impedance.

By way of example as illustrated FIG. 6, electrical impedance across two electrodes placed on either side of the micropillar array can be measured before and after sample perfusion. The microfluidic device consists of six micropillar arrays comprising microcapillaries from 12 μm down to 3 μm, which mimic key dimensions of small blood vessels observed in the capillary bed. The 12-μm array is designed to trap potential large-cell aggregates that may cause microchannel clogging. Prior to the measurement, PBS can be perfused through the microchannel at 100 mBar for 15 min to allow for any volumetric changes of the microchannel. Thereafter, the baseline impedance reading can be obtained for each micropillar array without stopping the PBS flow. Next, the RBC sample can be loaded into the sample reservoir and perfused for 20 min, followed by post-perfusion PBS washing for 20 min. A second impedance reading can then be obtained for each micropillar array upon the conclusion of the post-perfusion wash step. The resultant impedance change depends on microcapillary occlusion in the array.

In some embodiments, the processor can determine cumulative percentage impedance changes of pairs of electrodes flanking at least one micropillar array by comparing the impedance of the pair of electrodes before and after a blood sample is perfused through the microchannel. Cumulative percentage impedance changes can be determined using an algorithm of red blood cell (RBC) Electrical Impedance Index (REI) that is indicative of RBC pathology or occlusion. The algorithm can be integrated into or with the processor or control unit 24 of the microfluidic system.

The REI is based on the following equation:

$$REI = {\sum }_{i}^{n} \frac{{I}_{i 2} - {I}_{i 1}}{{I}_{i 1}} \times 100 \%$$

where,
n=total number of capillary networks within an area of interest;
i=the i^{th} capillary network within the area of interest;
Iᵢ₁=a first electrical impedance reading of an i^{th} capillary network; and
Iᵢ₂=a second electrical impedance reading of the i^{th} capillary network.

The REI can be indicative of RBC-mediated microvascular occlusion and serve as an in vitro therapeutic efficacy benchmark for assessing clinical outcome of RBC targeted and curative therapies.

In some embodiments, the processor can provide the same weight to electrical impedances measured from different pairs of electrodes. For example, the measured impedance of a pair of electrodes upstream in the microchannel can be weighted the same as a pair of electrodes downstream in the microchannel. The impedance measurements can be cumulated and compared to control impedance measurements to provide the cumulative percentage impedance changes.

In some embodiments, the microfluidic system can further include an imaging system 20 that can detect and measure through the at least one optically transparent wall the morphology and/or quantity of adhered, and/or captured cells of interest within each microchannel and optionally the viscosity of the fluid sample. The imaging system 20 can be a lens-based imaging system, lensless imaging system, and/or mobile imaging system, e.g., cellular phone camera. The imaging system 20 can include a control unit (not shown), which can a include a computer readable storage unit and a processor to analyze the images of the microchannels and provide real-time feedback to a subject of the results of the image acquisition/analysis. The control unit can be shared with the control unit 24 that configured to compare and/or determine changes of measured impedances of pairs of electrodes 42.

In some examples, the imaging system can be a lens-based imaging system or a lensless/mobile imaging system. In some embodiments, the lensless imaging system can be a CCD sensor and a light emitting diode. By way of example, a fluorescent microscopy camera (EXi Blue EXI-BLU—R-F-M-14-C) and an Olympus IX83 inverted, fluorescent motorized microscope with Olympus Cell Sense live-cell imaging and analysis software can be used to obtain real-time microscopic images. Olympus (20×/0.45 ph2 and 40×/0.75 ph3) long working distance objective lenses can be utilized for phase contrast imaging of cells occluded and/or adhered in the microchannels. During real-time microscope imaging and high resolution video recording at 10 fps rate, controlled fluid flow with stepwise increments can be applied until cell detachment from the microchannel surface is observed. Videos can be converted to single frame images for further processing and analysis. The cell dimensions can then analyzed by using Adobe Photoshop software (San Jose, CA).

In some examples, a mobile imaging and quantification algorithm can be integrated into or with the microfluidic device. The algorithm can achieve reliable and repeatable test results for data collected in all resource settings of the microfluidic device.

In other examples, the microfluidic device can be configured to cooperate with a cellular phone having imaging capabilities. In such a case, the cellular phone can be provided with or capable of obtaining image analysis algorithms/software, e.g., via an online application. Images can be recreated by the cellular phone camera software and loaded into a custom phone application that identifies occluded and/or adhered cells, such as RBCs, quantifies the number of occluded and/or adhered cells, such as RBCs, in the image, and displays the results.

The cells of interest can be blood cells obtained from the subject and the imaging system can quantify the adhered cells in the microchannel to measure the cell deformability and/or adherence. In other examples, the imaging system can quantify adhered cells in the microchannel to monitor the progression of a disease, such as SCD, of a subject from which the cells are obtained. In still other examples, the imaging system can quantify the occluded and/or adhered cells in each channel to measure the efficacy of a therapeutic treatment administered to a subject from which the cells are obtained.

In some embodiments, the imaging system 20 can be configured to provide particle image velocimetry of fluid in the microchannels. For example, the imaging system 20 can be configured to take images of fluid as it passes through an imaging field of the microchannel. These images can be sent to control unit that includes a computer readable storage medium for storing the images and a processor that include executable instructions for receiving sequential images, generating general velocity vector maps based on successive images, and generating mean flow velocity data from the velocity vector maps. The mean flow velocity data can be output from the processor to a display as raw data or as visual representation of the mean flow velocity. The mean flow velocity data or map can be correlated to viscosity of the fluid using the processor or another processor that outputs the viscosity date of the fluid as raw data or as visual depiction.

The image processing may be implemented using hardware, software or a combination thereof. When implemented in software, the software code can be executed on any suitable processor or collection of processors, whether provided in a single computer or distributed among multiple computers. Such processors may be implemented as integrated circuits, with one or more processors in an integrated circuit component. Though, a processor may be implemented using circuitry in any suitable format.

In some embodiments, the microfluidic system 10 can further includes a micro-gas exchanger (not shown) fluidly connected to the at least one microchannel 16 for varying the oxygen content of the fluid sample containing the cells. In one embodiment, the micro-gas exchanger can include a gas-permeable inner tube inserted within a gas-impermeable outer tube. Fluid, such as blood, containing the cells of interest can be delivered through the inner tube such that the fluid exchanges gases through the permeable tubing wall with a control gas, e.g., 5% CO₂ and 95% N₂, between the tubes. The oxygen content of the fluid exiting the micro-gas exchanger is controlled to thereby control the oxygen content of the fluid delivered to the microchannel.

By way of example, the micro-gas exchanger can include concentric inner and outer tubes. The inner tube has a gas-permeable wall defining a central passage extending the entire length of the inner tube. The outer tube has a gas impermeable wall defining a central passage extending the entire length of the outer tube. An annular space is formed between the tubes. The central passage receives the fluid sample and is in fluid communication with one or more inlet ports of the microfluidic device. Each inlet port can be fluidly connected to the same micro-gas exchanger or a different micro-gas exchanger to specifically tailor the fluid delivered to each microchannel. An outlet tube is connected to each outlet port of the micro-gas exchanger. A controlled gas flow takes place in the annular space between the concentric tubes and fluid flows inside the inner tube. When the fluid sample is blood, deoxygenation of the sample occurs due to gas diffusion (5% CO₂ and 95% N₂) through the inner gas-permeable wall.

In another embodiment, the micro-gas exchanger can be integrated with the housing and the at least one microchannel 16 to provide a chamber for varying the oxygen content of the fluid sample containing the cells. In one embodiment, the micro-gas exchanger can include a gas-permeable inner wall inserted within a gas-impermeable outer wall. Fluid, containing the cells of interest can be delivered through the microchannel such that the fluid exchanges gases through the permeable wall with a control gas, e.g., 5% CO₂ and 95% N₂, between the walls. The oxygen content of the fluid exiting the micro-gas exchanger is controlled to thereby control the oxygen content of the fluid delivered to the microchannel.

By way of example, the housing can include overlapping inner and outer walls. The inner wall is gas-permeable wall defines the microchannel through the housing. The outer wall is gas impermeable and defines a central passage extending the entire length of the outer wall and inner wall. A space is formed between the inner and outer walls. The microchannel receives the fluid sample from one or more inlet ports of the microfluidic device. An outlet tube is connected to outlet port of the micro-gas exchanger. A controlled gas flow takes place in the space between the outer and inner walls and fluid flows inside the microchannel. When the fluid sample is blood, deoxygenation of the sample occurs due to gas diffusion (5% CO₂ and 95% N₂) through the inner gas-permeable wall.

It can be expected that a microfluidic device and system described herein is applicable to the study or simulation of cell heterogeneity, deformability, and adherence as well as microvascular occlusion within subjects in larger clinically diverse populations and may provide important insights into complex disease phenotypes. For example, abnormal RBC deformability and/or adhesion to microvascular surfaces has previously been implicated in multi-system diseases, such as sickle cell disease (SCD), β-thalassemia, diabetes mellitus, hereditary spherocytosis, polycythemia vera, and malaria.

By way of example, sickled RBC occlusion or adherence to blood vessel walls has been shown to take place in post-capillary venules. To this end, this application contemplates a microfluidic testing method utilizing pathophysiologic correlates, including but not limited to, analyses of deformability or adhesion of RBCs as well as RBC-mediated microvascular occlusion, at baseline and during vaso-occlusive crises, with treatment, and in the presence of end-organ damage. In some examples, the testing method provides a highly specific analyses of the properties of RBCs, WBCs, circulating hematopoietic precursor cells and circulating endothelial cells. In one example, the testing method is performed using a miniscule blood sample (<15 μL). The testing method can provide a sophisticated and clinically relevant strategy with which patient blood samples and/or blood cells may be serially examined for cellular/membrane/adhesive properties during disease progression.

The microfluidic system can evaluate membrane and cellular abnormalities by interrogating a number of recognized abnormalities in a range of clinical phenotypes. To date, these phenotypes are discussed in various correlative blood cell studies ranging between clinical reports, testing results, interventions, and/or chart reviews.

The system described herein has advantages because existing conventional methods cannot assess longitudinal and large-scale blood cell clinical correlations with cellular, membrane, and adhesive properties. To this end, this application contemplates a method for using the microfluidic system for examining cellular properties and interactions as well as vascular occlusion. These cellular properties and interactions include, but are not limited to, RBC cellular, adhesive, and occlusive properties, WBC cellular and adhesive properties, circulating endothelial characteristics, hematopoietic precursor cell characteristics, and vascular occlusion. A simple test for blood cell deformability and adhesion or microvascular occlusion allows exploration of its role in chronic complications in SCD, in addition to during crisis.

In some embodiments, the microfluidic system can be used in methods for analyzing, characterizing and/or predicting the deformability of cells, such as RBCs and WBCs, as well as the adherence of such cells to various capturing agents, such as such as laminin, fibronectin, E-Selectin, P-Selectin, L-selectin, intracellular adhesion molecule 1 (ICAM-1), or vascular cellular adhesion molecule 1 (VCAM-1), provided in the microchannel of the microfluidic device. In further embodiments, methods and devices are provided for diagnosing, assessing, characterizing, evaluating, and/or predicting disease based on the deformability of the cells or adherence of the cells to the capturing agents in microchannels as well as the viscosity of a fluid sample, such as blood.

Any appropriate condition or disease of a subject may be evaluated using the methods, systems, and devices described herein, typically provided that a cell may be obtained from the subject that has a material property (e.g., deformability, adherence, etc.) that is indicative of the condition or disease. The condition or disease to be detected may be, for example, a hematological disorder, such as hematological cancer, anemia, infectious mononucleosis, HIV, malaria, leishmaniasis, sickle cell disease (SCD), babesiosis, spherocytosis, monoclonal gammopathy of undetermined significance or multiple myeloma. Examples of hematological cancer include, but are not limited to, Hodgkin's disease, Non-Hodgkin's lymphoma, Burkitt's lymphoma, anaplastic large cell lymphoma, splenic marginal zone lymphoma, hepatosplenic T-cell lymphoma, angioimmunoblastic T-cell lymphoma (AILT), multiple myeloma, Waldenstrom macroglobulinemia, plasmacytoma, acute lymphocytic leukemia (ALL), chronic lymphocytic leukemia (CLL), B cell CLL, acute myelogenous leukemia (AML), chronic myelogenous leukemia (CML), T-cell prolymphocytic leukemia (T-PLL), B-cell prolymphocytic leukemia (B-PLL), chronic neutrophilic leukemia (CNL), hairy cell leukemia (HCL), T-cell large granular lymphocyte leukemia (T-LGL) and aggressive NK-cell leukemia. The foregoing diseases or conditions are not intended to be limiting. It should thus be appreciated that other appropriate diseases or conditions may be evaluated using the methods disclosed herein.

Methods are also provided for detecting and characterizing a leukocyte-mediated condition or disease. For example, methods are provided for detecting and characterizing a leukocyte-mediated condition or disease associated with the lungs of a subject being highly susceptible to injury, possibly due to activated leukocytes with altered deformability, having altered ability to circulate through the pulmonary capillary bed. Methods such as these, and others disclosed herein, can also be applied to detect and/or characterize septic shock (sepsis) that is associated with both rigid and activated neutrophils. Such neutrophils can, in some cases, occlude capillaries and damage organs where changes in neutrophil cytoskeleton are induced by molecular signals leading to decreased deformability.

In some embodiments, methods described herein can provide measurement of adhesion of a cell population or cell-mediated microvascular occlusion by measuring the impedance of the micropillar arrays of the microchannel. The measured impedance of micropillar arrays of the microchannels can be used to generate an RBC electrical impedance index (REI) that is indicative of RBC-mediated microvascular occlusion of the RBCs and increased risk of vaso-occlusive crises (VOC) and/or microvascular health and function. By way of example, the REI can be equal to the summation of the measured number of occluded microcapillaries in one micropillar array. In some embodiments, the measured REI can be compared to a control value. The subject can have an increased risk of vaso-occlusive crises (VOC) and/or decreased microvascular health and function when the REI is greater than the control value.

A “control value” or “appropriate standard” is a standard, parameter, value or level indicative of a known outcome, status or result (e.g., a known disease or condition status). A control value or appropriate can be determined (e.g., determined in parallel with a test measurement) or can be pre-existing (e.g., a historical value, etc.). For example, a control value or appropriate standard may be the REI of cells obtained from a subject known to have a disease, or a subject identified as being disease-free. In the former case, a lack of a difference between the measured REI and the REI of an appropriate standard may be indicative of a subject having a disease or condition. Whereas in the latter case, the presence of a difference between the measured REI and the REI of the control value or appropriate standard may be indicative of a subject having a disease or condition. While the control value or appropriate standard is described herein as being based on REI, the control value or appropriate is not so limited and can include any mechanical property or rheological property of a cell obtained from a subject who is identified as not having the condition or disease or can be a mechanical property or rheological property of a cell obtained from a subject who is identified as having the condition or disease.

The magnitude of a difference between a parameter, level or value and an appropriate standard that is indicative of known outcome, status or result may vary. For example, a significant difference that indicates a known outcome, status or result may be detected when the level of a parameter, level or value is at least 1%, at least 5%, at least 10%, at least 25%, at least 50%, at least 100%, at least 250%, at least 500%, or at least 1000% higher, or lower, than the appropriate standard. Similarly, a significant difference may be detected when a parameter, level or value is at least 2-fold, at least 3-fold, at least 4-fold, at least 5-fold, at least 6-fold, at least 7-fold, at least 8-fold, at least 9-fold, at least 10-fold, at least 20-fold, at least 30-fold, at least 40-fold, at least 50-fold, at least 100-fold, or more higher, or lower, than the level of the appropriate standard. Significant differences may be identified by using an appropriate statistical test. Tests for statistical significance are well known in the art and are exemplified in Applied Statistics for Engineers and Scientists by Petruccelli, Chen and Nandram Reprint Ed. Prentice Hall (1999).

Further, certain methods described herein provide for measurement of adhesive properties of a cell population, in combination with or separate from measurement of the cell deformability or cell-mediated occlusion. The combination of determining cytoadhesive properties and the deformative properties of a cell population, particularly a cell population containing a plurality of different cell types (e.g., RBCs and WBCs), may be used to generate a “Health Signature” that comprises an array of properties that can be tracked in a subject over a period of time. Such a Health Signature may facilitate effective monitoring of a subject's health over time. Such monitoring may lead to an early detection of potential acute or chronic infection, or other disease, disorder, fitness, or condition. In some cases, further, knowledge of the overall rheology of a material, along with either the deformative or cytoadhesive property of a cell, allows the determination of the other property.

In some embodiment, the cell-mediated occlusion and/or adherence of cells perfused through the microchannel of the microfluidic device can be used for evaluating, assessing, monitoring, and/or predicting disease status, disease prognosis, treatment course (e.g., therapeutic selection, dosing schedules, administration routes, etc.), response to treatment and/or treatment efficacy.

In some embodiments, the microfluidic device described herein can be used to assess the health of any of the subjects described herein, used to detect or determine the stage of any of the diseases or conditions described herein and can be used for determining the number of diseased versus healthy cells.

In other embodiments, a method for detecting a condition or disease in a subject can include obtaining cells, such as a RBCs, WBCs, stem cells, or plasma cells, from the subject and perfusing a fluid containing the cells through the microfluidic channel that includes the microcapillaries and optionally various capturing agents provided in or functionalized to the microchannels.

The cells can be obtained directly or indirectly by acquiring a biological sample from a subject. For example, a biological sample may be obtained (e.g., at a point-of-care facility, e.g., a physician's office, a hospital, laboratory facility) by procuring a tissue or fluid sample (e.g., blood draw, marrow sample, spinal tap) from a subject. Alternatively, a biological sample may be obtained by receiving the biological sample (e.g., at a laboratory facility) from one or more persons who procured the sample directly from the subject. The biological sample may be, for example, a tissue (e.g., blood), cell (e.g., hematopoietic cell such as hematopoietic stem cell, leukocyte, or reticulocyte, stem cell, or plasma cell), vesicle, biomolecular aggregate or platelet from the subject.

The cell-mediated occlusion and/or adherence of cells, such as RBCs and WBCs in the microchannels of the microfluidic device can then be determined by measuring the REI and the REI can be compared to a standard or control to indicate whether the subject has the condition or disease; and optionally, diagnosing the subject as having the condition or disease based on the results. The appropriate standard or control can be the REI that was obtained from a subject who is identified as not having the condition or disease. The REI can also be measured and compared to a control or standard to indicate or further characterize whether the subject has the condition or disease.

Other embodiments described herein relate to a method of assessing microvascular health and function of a subject in need thereof. The method can include perfusing a fluid sample including RBCs from the subject through the at least one microchannel of a microfluidic device described herein. The impedance of the micropillar arrays in the at least one microchannel can then be measured. A RBC electrical impedance index (REI) can be generated from the measured impedance of occluded microcapillaries of the micropillar arrays. The REI can be indicative of increased risk of vaso-occlusive crises (VOC) and/or microvascular health and function

In some embodiments, the REI can be compared to a control value. The subject can have an increased risk of vaso-occlusive crises (VOC) and/or decreased microvascular health and function when the REI is greater than the control value.

In some embodiments, the fluid sample can be perfused under at least one normoxic or hypoxic conditions and the number of RBCs can be measured using an imaging system.

Still other embodiments relate to a method of assessing the pathology of RBCs. The method can include perfusing a fluid sample including the RBCs through the at least one microchannel of a microfluidic device described herein. The number of microcapillaries occluded with RBCs can be measured by measuring the impedance of the micropillar arrays in the at least one microchannel. A RBC occlusive index (REI) can be generated from the measured impedance of the micropillar arrays. The REI can be indicative of the number of pathologically impaired RBCs.

In some embodiments, the RBCs are from a subject at risk of a vaso-occlusive crises and/or decreased microvascular health and function. The subject has an increased risk of vaso-occlusive crises (VOC) and/or decreased microvascular health and function when the REI is greater than the control value. In some embodiments, subject can have or be at an increased risk of malaria or sickle cell disease. The RBCs can be from stored blood and/or blood to be transfused and the REI can be used to determine the fitness or storage lesions of the stored RBCs and/or RBCs to be transfused.

Other embodiments described herein relate to a method of measuring efficacy of a therapeutic agent in modulating blood cell adhesion and/or deformability. The method can include provide a microfluidic device as described herein and perfusing a fluid sample including blood cells through the at least one microchannel of the microfluidic device. The impedance of at least one micropillar array in the at least one microchannel can be measured after the fluid sample is perfused through the microchannel. The therapeutic agent can be added to at least one of the fluid sample prior to perfusion through the at least one microchannel. The efficacy of the therapeutic agent can be determined based on the measured impedance of the at least one micropillar array. A decrease in the measured impedance compared to a control is indicative of the therapeutic agent having an increased efficacy in decreasing blood cell adhesion and/or increasing blood cell deformability.

In some embodiments, the fluid sample can be perfused under at least one of normoxic or hypoxic conditions.

Other embodiments described herein relate to a method of assessing clinical outcome of a subject administered a RBC therapy. The method can include providing a microfluidic device as described herein and perfusing a fluid sample including RBCs from the subject administered the RBC therapy through the at least one microchannel of the microfluidic device. The impedance of at least one micropillar array in the at least one microchannel can be measured after the fluid sample is perfused through the microchannel. The clinical outcome of the subject can be determined by comparing the measured impedance of the at least one micropillar array to a control.

In some embodiments, a difference in the measured impedance compared to a control is indicative of a favorable clinical outcome.

In other embodiments, a difference in the measured impedance compared to a control is indicative of an unfavorable clinical outcome.

### Example 1

In this example, we describe a standardized, functional in vitro microfluidic assay that allows objective and quantitative assessment of RBC mediated microvascular occlusion. To that end, we designed a microfluidic device with embedded micropillar arrays comprising a gradient of narrow microcapillaries from 12 μm down to 3 μm along the flow direction mimicking the non-uniform small blood vessels in the capillary bed. Such design enabled stiffer RBCs to be retained in the upstream arrays representing larger microcapillaries, while less stiff RBCs were retained in the downstream arrays representing smaller microcapillaries. The upstream array with 12-μm microcapillaries, which does not represent the typical microcapillary dimension in the capillary bed (which are typically less than 10 μm), was included to trap potential large-cell aggregates. Moreover, the micropillar arrays were coupled with two 40-μm-wide side pathways that mimic arteriovenous anastomoses. Anastomoses are bypass passageways around capillary beds, which provide alternative flow paths in the event of blockages in the microcapillaries. These anastomosis-mimicking pathways helped regulate blood flow such that when the upstream portion of an array was saturated, the incoming RBCs could still flow around and into the downstream arrays, which prevented congestion of the microchannel. These features enabled testing of clinical samples at near-hematocrit levels and full utilization of the microcapillary domain. Finally, each micropillar array was paired with two sputtered gold electrodes on the channel bottom surface for electrical impedance measurement. The impedance of each array across the paired electrodes was obtained before and after sample perfusion. We used healthy RBCs, glutaraldehyde-stiffened RBCs, and RBCs from two common red cell disorders, SCD and HS, to validate the microfluidic design and to demonstrate its clinical relevance. We introduced two new parameters, ‘RBC Occlusion Index’ (ROI) and ‘RBC Electrical Impedance Index’ (REI), which represent the cumulative percentage occlusion and cumulative percentage impedance change, respectively. We showed that the REI significantly correlates with the ROI, and both ROI and REI associate with in vivo hemolytic biomarkers, serum lactate dehydrogenase (LDH) levels and absolute reticulocyte counts (ARCs), as well as treatment outcomes in subjects with SCD. The presented microfluidic device with the integrated electrical impedance measurement, in which the need for high-resolution imaging is obviated, enables future translation of this technology for widespread access and point-of-care use.

### Methods

### Concept and Design

The microfluidic design comprises capillary network-inspired micropillar arrays and sputtered electrodes paired with each array. As RBCs flow through the microchannel, deformable RBCs are able to clear through, while stiff RBCs are retained within the microcapillaries (FIG. 6A). As a result, the electrical impedance of the micropillar array across the paired electrodes increases in accordance with the resultant occlusion due to the retained RBCs. The microchannel geometry features two key aspects of the human capillary bed: the small capillaries (less than 10 μm) and the arteriovenous anastomoses. To mimic small capillaries, micropillar arrays were embedded into the microchannel comprising microcapillaries from 12 μm down to 3 μm along the flow direction (FIG. 6B). The micropillars were designed to be 20-μm long, 10-μm wide, and 12-μm tall (FIG. 6B inset), with a column-to-column spacing of 20 μm and array-to-array spacing of 1 mm. This feature enabled less stiff RBCs to be retained by downstream finer microcapillaries while stiffer RBCs were retained by upstream coarse microcapillaries. The near-inlet 12-μm micropillar array was designed to retain large-cell aggregates or contamination that may be present in the blood flow. To mimic the anastomoses around capillary beds, the micropillar arrays were coupled with two 40-μm-wide side pathways (FIG. 6B inset). This feature helped regulate blood flow around the obstructed area to prevent clogging of the microchannel. Moreover, each micropillar array was flanked by a pair of planar gold electrodes for sensing variation of electrical impedance due to retained RBCs (FIG. 6B). Overall, the microchannel is 24-mm long, 4-mm wide, and 12-μm thick.

### Device Fabrication

The microfluidic device was fabricated using standard soft photolithography protocols. Initially, a master silicon wafer was fabricated by micropatterning a uniform SU8-2010 (Microchem, Newton, MA) photoresist layer. Briefly, the SU8-2010 layer was spin-coated at 2500 rpm over the silicon wafer and soft-baked at 95° C. for 4 min. Next, the wafer was exposed to UV light and post-exposure baked at 95° C. for 4 min. Thereafter, the wafer was developed in 1-methoxy-2-propanol acetate (Sigma Aldrich, St. Louis, MO), and hard-baked at 110° C. overnight. The master wafer was then used for PDMS casting (10:1 ratio, 80° C. overnight). The PDMS blocks were peeled-off and punched with 0.5 mm-diameter ports for inlets and outlets. Substrate fabrication process started with sonicating standard microscope glass slides with isopropanol for 15 min at room temperature. After drying, multiple gold electrodes with dimensions of 10.5 mm×0.5 mm with a spacing of 2.5 mm and contact pads with dimensions of 4.5 mm×4.5 mm were sputter-deposited (150 A°/2000 A° Ti/Au) under a laser-micromachined Kapton tape mask. Thereafter, a layer of amorphous silica (5000 A°/SiO₂) was sputter-deposited under a secondary laser-micromachined Kapton tape mask to ensure proper sealing. All radiofrequency (RF) sputtering processes were performed in Denton Vacuum Explorer 14 System (Moorestown, NJ). Finally, the PDMS block was covalently bonded to the substrate using oxygen plasma. The obtained device was incubated at 60° C. for 30 min on a hotplate to increase the bonding strength. Detailed fabrication process of the substrate is illustrated in FIG. 11. The cross marks were designed to achieve electrode and micropillar array alignment.

### Blood Sample Acquisition and Processing

De-identified blood samples were collected in ethylenediaminetetraacetic acid (EDTA)-containing vacutainers from healthy donors or subjects with homozygous (HbSS) SCD or HS at University Hospitals Cleveland Medical Center under Institutional Review Board (IRB)-approved protocols. The obtained blood samples were stored at 4° C. until tested. Signed informed consent forms were obtained from all study participants. RBCs were isolated from the obtained whole blood samples by centrifuging at 500×g for 5 min at room temperature. Plasma, buffy coat, and the near-plasma portion of the RBC pellet were carefully removed. The RBC pellet was then washed twice in phosphate-buffered saline (PBS, 1X). Unless otherwise stated, the isolated RBCs were re-suspended in PBS at 20% hematocrit and tested. All experiments were completed within 48 hours of venipuncture in this study. Clinical variables of the study population with SCD are summarized in Table 1.

TABLE 1
Clinical variable of the study population with SCD

| Clinical variables | Range | Mean + SD |
|---|---|---|
| ROI (%) | 9.26-66.63 | 34.65 ± 21.99 |
| REI (%) | 2.44-32.01 | 14.93 ± 10.48 |
| Age (years) | 20-55 | 37 ± 10 |
| Hemoglobin (g/dL) | 6.2-12.1 | 8.56 ± 1.69 |
| Hematocrit (%) | 18.0-37.1 | 25.6 ± 5.1 |
| White Blood Cell Count (10⁹/L) | 6.9-18.2 | 12.5 ± 3.5 |
| Platelet Count (10⁹/L) | 130-528 | 346 ± 143 |
| Absolute Neutrophil Count (10⁶/L) | 2360-12030 | 7526 ± 2738 |
| Absolute Reticulocyte Count (10⁹/L) | 48-732 | 327 ± 194 |
| Lactate Dehydrogenase (U/L) | 192-490 | 299 ± 97 |
| Ferritin (μg/L) | 32-7640 | 3119 ± 2379 |
| Hemoglobin S (%) | 6.2-86.5 | 44.3 ± 24.7 |
| Hemoglobin A (%) | 5.2-74.9 | 42.5 ± 22.1 |
| Hemoglobin F (%) | 0.3-10.1 | 2.8 ± 3.0 |
| Subjects on Transfusion | 67% (8 out of 12) | |

A total of 12 blood samples were obtained from 12 subjects with homozygous SCD (HbSS, total N = 12, Male = 5, Female = 7).
ROI: RBC Occlusion Index.
REI: RBC Electrical Impedance Index.
SD: Standard deviation.

### Glutaraldehyde Stiffening of Healthy RBCs

In order to validate the microfluidic device functionality of assessing RBC mediated microvascular occlusion, healthy RBCs were isolated and treated with 0.08% w/v glutaraldehyde (Sigma Aldrich) for 10 min at room temperature. The glutaraldehyde-stiffened RBCs were then washed with PBS, mixed with untreated healthy RBCs from the same donor at either 1% or 2% v/v ratio, and re-suspended in PBS at 20% hematocrit for testing. Healthy RBCs from the same healthy donor were included in the study design as control.

### Test Procedure

Microchannels were initially washed with absolute ethanol (100%) and PBS, which was followed by incubation with 2% bovine serum albumin (BSA) overnight to prevent non-specific binding of RBCs to the channel walls. A Flow-EZ™ microfluidic flow control system (Fluigent, Lowell, MA) was used to regulate the flow (FIG. 12). An impedance analyzer (Agilent 4294A, Santa Clara, CA) coupled with a custom printed-circuit board was used to perform electrical impedance measurements. Briefly, impedance magnitude across each of the 3-μm to 10-μm micropillar arrays was recorded over the frequency range of 40 Hz-1 MHz before introducing blood into the microchannel and after completing the washing step. FIG. 13 shows the raw impedance data of the 3-μm micropillar array measured for a typical clinical blood sample. For all impedance analyses, a spot frequency of 10 kHz was chosen to minimize potential electrode polarization effects at lower frequencies and avoid parasitic inductances associated with higher frequencies. Hence, data are reported as a percentage change of impedance at 10 kHz in this study.

Prior to the experiment, PBS was perfused through the microchannel at 100 mBar for 15 min to allow for any volumetric changes of the microchannel. Thereafter, the baseline impedance reading was obtained for each micropillar array without stopping the PBS flow. Next, the RBC sample was loaded into the sample reservoir and perfused for 20 min, followed by post-perfusion PBS washing for 20 min. A second impedance reading was then obtained for each micropillar array upon the conclusion of the post-perfusion wash step. The 12-μm micropillar array was excluded from the measurement since it was designed to prevent large-cell aggregates or contamination, as stated earlier. System background noise was characterized by repeatedly testing PBS (background electrolyte) using five different devices. For select experiments, the electrical impedance of the 3-μm micropillar array was continuously monitored over the entire duration of the experiment.

To determine the association between the electrical impedance change and microcapillary occlusion in the microchannel, an Olympus IX83 inverted motorized microscope with Olympus CellSense live-cell imaging and analysis software were used to obtain high-resolution microscopic images. The images were further processed by Adobe Photoshop software (San Jose, CA), in which the obstructed microcapillaries were manually counted. Typically, it takes less than 45 min to complete the microfluidic processing and electrical impedance data acquisition/analysis for testing one clinical blood sample. The microfluidic device was designed to be single-use and disposable to prevent any cross-contamination between the samples tested. Each data point in this manuscript was generated with a single use of a newly made device.

### RBC Occlusion Index and RBC Electrical Impedance Index

To effectively compare the overall microcapillary occlusion and the overall impedance change caused by different RBC samples, we defined ‘RBC Occlusion Index’ (ROI) and ‘RBC Electrical Impedance Index’ (REI) using the following equations:

$$ROI = {\sum }_{i}^{n} \frac{{O}_{i}}{{N}_{i}} \times 100 \% \qquad ( 1 )$$

$$REI = {\sum }_{i}^{n} \frac{{I}_{i 2} - {I}_{i 1}}{{I}_{i 1}} \times 100 \% \qquad ( 2 )$$

where,
n=Total number of capillary networks within the area of interest
i=The i^{th} capillary network within the area of interest
Oᵢ; =The number of microcapillary occlusion in the i^{th} capillary network
Nᵢ=Total number of microcapillaries in the i^{th} capillary network
Iᵢ₁=The first electrical impedance reading of the i^{th} capillary network
Iᵢ₂=The second electrical impedance reading of the i^{th} capillary network

Therefore, ROI represents the cumulative percentage occlusion of the capillary networks and REI represents the cumulative percentage impedance change of the capillary networks across the device. The area of interest in our device contains the 3-μm to 10-μm micropillar arrays. The upstream 12-μm array was included to trap potential large-cell aggregates. The concepts of ROI (i.e., the cumulative percentage occlusion of the capillary networks) and REI (i.e., the cumulative percentage impedance change of the capillary networks) are translatable to any microfluidic device employing capillary networks and electrical impedance measurement to assess occlusion/impedance change due to abnormal cellular deformability.

### Statistical Methods

Data were reported as mean±standard deviation (mean±SD) in this study. Data were initially analyzed for normality followed by appropriate comparison methods: paired t-test for paired data, one-way ANOVA for normally distributed data, or Mann-Whitney for non-normally distributed data. Linear regression was used to assess the relationship between two variables, and the Pearson correlation coefficient (PCC) was reported. Mean-shift clustering technique was used to categorize the study population with SCD based on their ROI and REI results. A custom-written code in MATLAB (MathWorks, Natick, MA) was utilized for the clustering analysis. Statistical significance was defined with p-value less than 0.05 (p<0.05). All statistical analyses were carried out using Minitab 19 software (Minitab Inc., State College, PA).

### Results

### Characterization of System Background Noise

We tested PBS (background electrolyte) using five different devices and found that the resultant electrical impedance changes of individual micropillar arrays ranged from −0.57% to 0.95% (FIG. 44, mean±SD=0.14%±0.33%). The 95% confidence interval was calculated as 0.01-0.27%. Hence, any impedance changes less than 0.27% could be attributed to the system background noise, and were thereby rounded to zero in blood tests.

### Validation of the Micropillar Arrays and the Integrated Electrical Impedance Measurement Using Glutaraldehyde-Stiffened RBCs

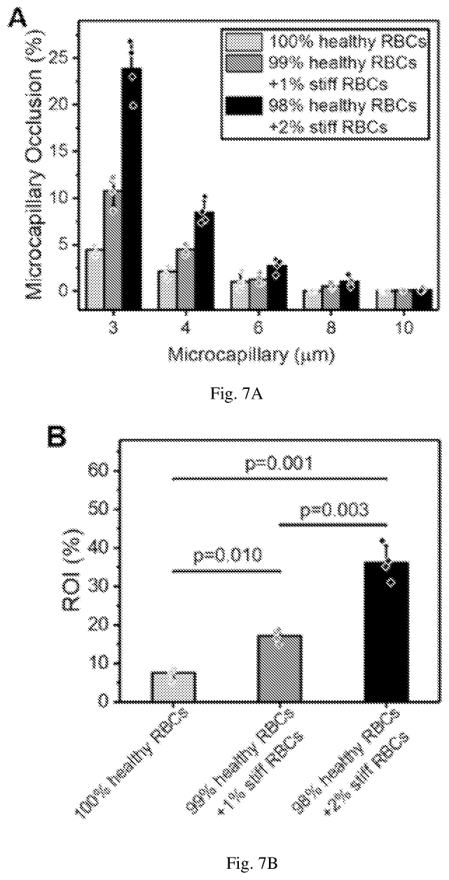

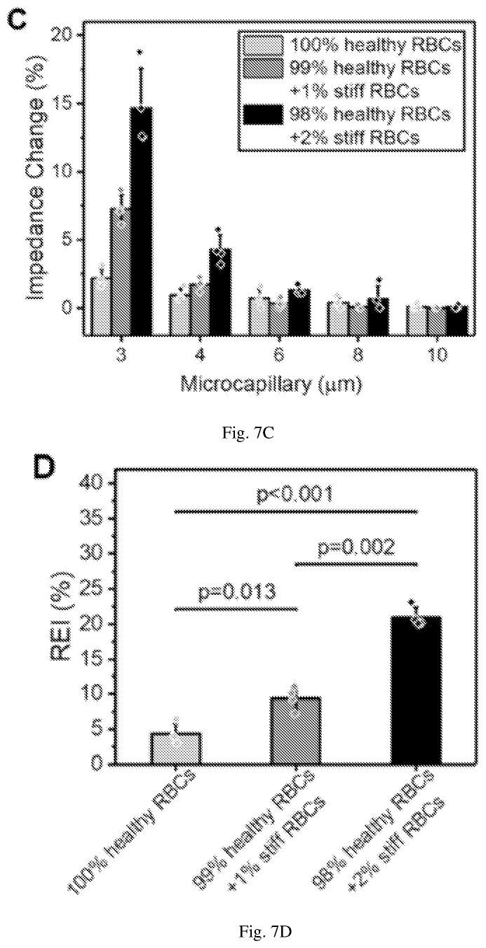

Glutaraldehyde is a non-specific protein cross-linker, which is commonly used to stiffen RBCs in order to mimic pathologically abnormal RBC deformability. Mild glutaraldehyde stiffening (0.08% w/v in PBS) was applied to RBCs in order to validate the micropillar arrays and the integrated electrical impedance measurement (FIG. 6C and insets). Of note, continuous monitoring of the impedance of the 3-μm micropillar array with four different samples, including PBS, a sample with 100% healthy RBCs, and a sample with 98% healthy RBCs and 2% stiff RBCs, revealed a unique feature of the presented device: the profile of the impedance change is significantly affected by the presence of healthy RBCs and stiff RBCs in the blood flow (FIG. 6D). In addition, we found that the level of microcapillary occlusion increased as the fraction of the stiff RBCs in the tested RBC samples increased (FIG. 7A). Further, the ROI of samples with 98% healthy RBCs and 2% stiff RBCs was significantly higher compared to that of samples with 99% healthy RBCs and 1% stiff RBCs or 100% healthy RBCs (FIG. 7B, mean±SD=36.16%+4.44% vs. 17.15%±1.78% or 7.61%±1.67% for 2% stiff RBCs vs. 1% stiff RBCs or healthy, p=0.003 or p=0.001, paired t-test), and the ROI of samples with 99% healthy RBCs and 1% stiff RBCs was significantly higher compared to that of 100% healthy RBCs (FIG. 7B, p=0.010, paired t-test). Notably, we observed that magnitude of impedance variation also increased as the fraction of the stiff RBCs in the tested RBC samples increased (FIG. 7C). Moreover, the REI of samples with 98% healthy RBCs and 2% stiff RBCs was significantly higher compared to that of samples with 99% healthy RBCs and 1% stiff RBCs or 100% healthy RBCs (FIG. 7D, mean±SD=20.99%±1.42% vs. 9.40%±1.61% or 4.35%±1.44% for 2% stiff RBCs vs. 1% stiff RBCs or healthy, p=0.002 or p<0.001, paired t-test), and the REI of samples with 99% healthy RBCs and 1% stiff RBCs was significantly higher compared to that of 100% healthy RBCs (FIG. 2D, p=0.013, paired t-test). Importantly, our results reveal that REI significantly correlates with ROI in these tests (FIG. 2E, PCC=0.987, p<0.0001, N=12).

### Validation of the Process Repeatability and Reproducibility of Results

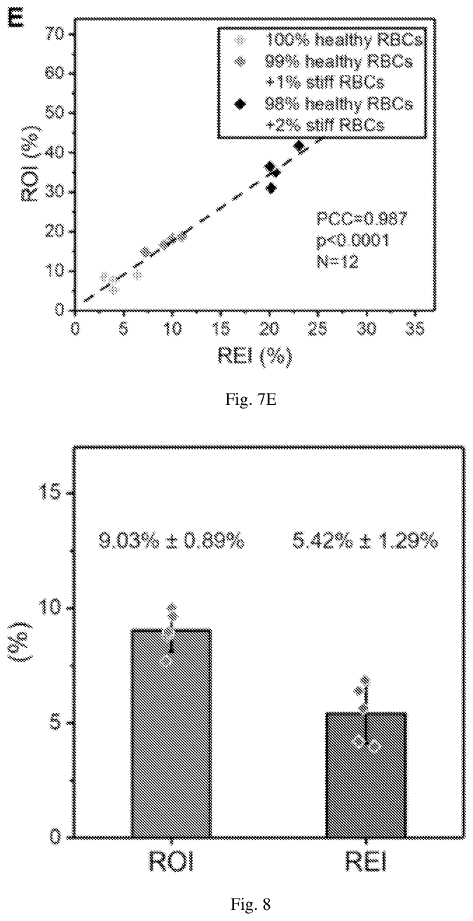

To validate the process repeatability and reproducibility of results, we tested one RBC sample obtained from a single healthy donor using five devices manufactured from different batches. The results were highly consistent, where the ROI of the sample is 9.03%±0.89%, and the REI of the sample is 5.42%±1.29% (FIG. 8, mean±SD).

### Assessments of RBC Mediated Microvascular Occlusion in Sickle Cell Disease and Hereditary Spherocytosis

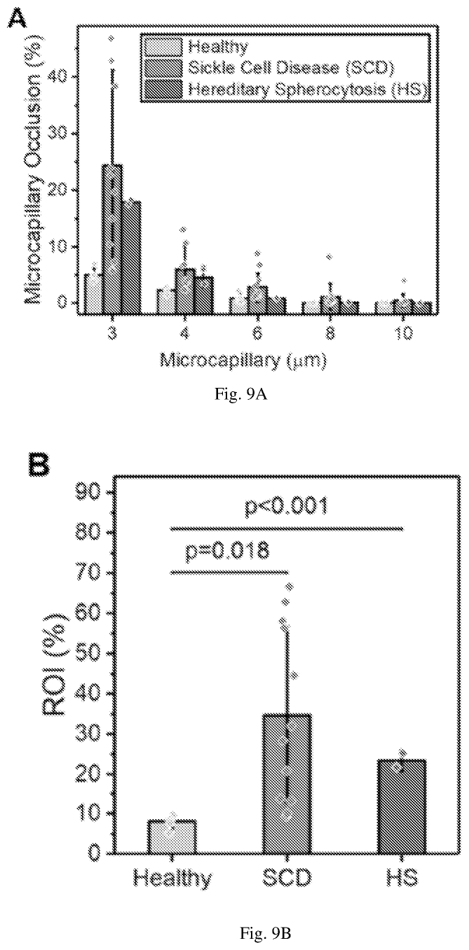

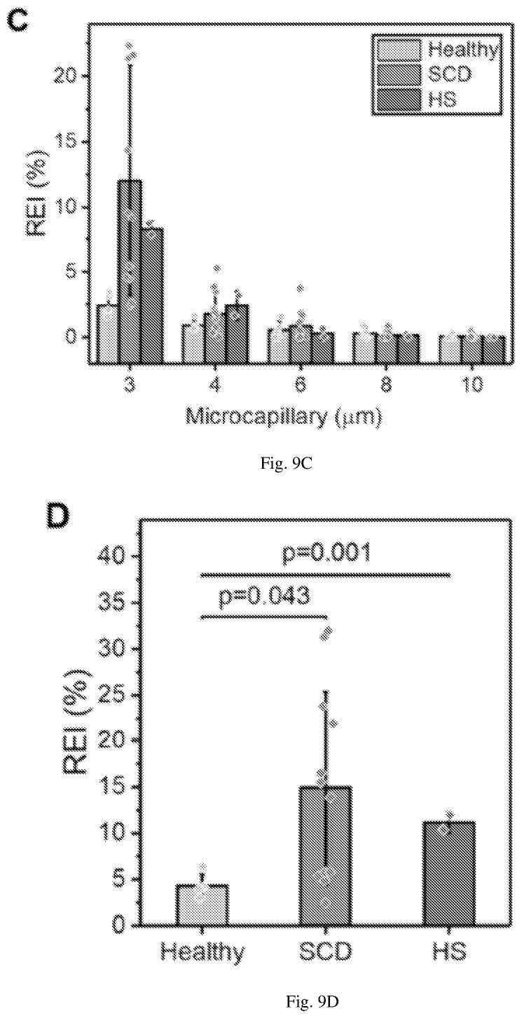

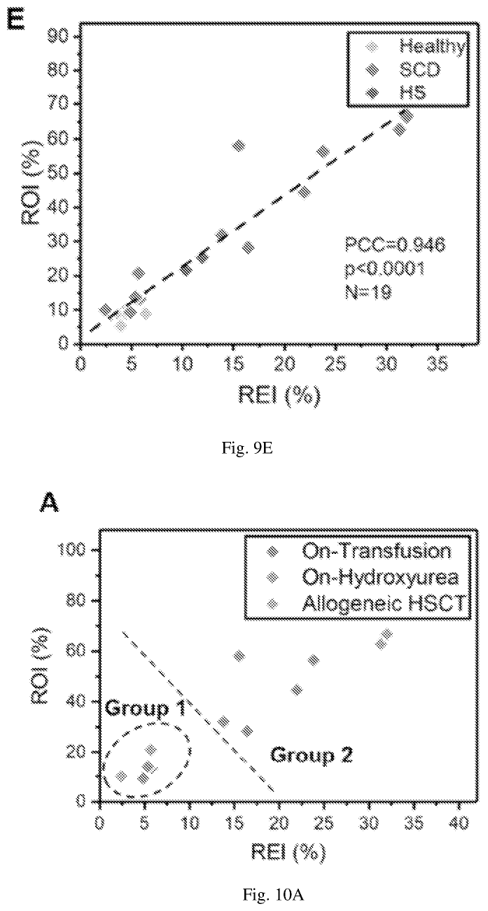

To demonstrate the clinical relevance of the present microfluidic device and the integrated electrical impedance measurement, we tested clinical samples from 12 subjects with homozygous (HbSS) SCD and 2 subjects with HS and compared the results with samples from 5 health donors. We found that the level of microcapillary occlusion increased when comparing RBCs from subjects with SCD or HS to RBCs from healthy donors (FIG. 9A). Further, the ROI of RBCs from subjects with SCD or HS is significantly higher compared to that of RBCs from healthy donors (FIG. 9B, mean±SD=34.65%±21.99% or 23.46%±2.55% vs. 8.02%±1.71% for SCD or HS vs. healthy, p=0.018 or p<0.001, one-way ANOVA). Notably, magnitude of impedance variation also increased when comparing RBCs from subjects with SCD or HS to RBCs from healthy donors (FIG. 9C). Moreover, the REI of RBCs from subjects with SCD or HS is significantly higher compared to that of RBCs from healthy donors (FIG. 9D, mean±SD=14.93%±10.48% or 11.17%±1.14% vs. 4.31%±1.25% for SCD or HS vs. healthy, p=0.043 or p=0.001, one-way ANOVA). Further, our results indicate that REI significantly correlates with ROI in these tests (FIG. 9E, PCC=0.946, p<0.0001, N=19).

Rbc Mediated Microvascular Occlusion and the Resultant ROI and REI Correlate with Clinical Hemolytic Biomarkers in Sickle Cell Disease

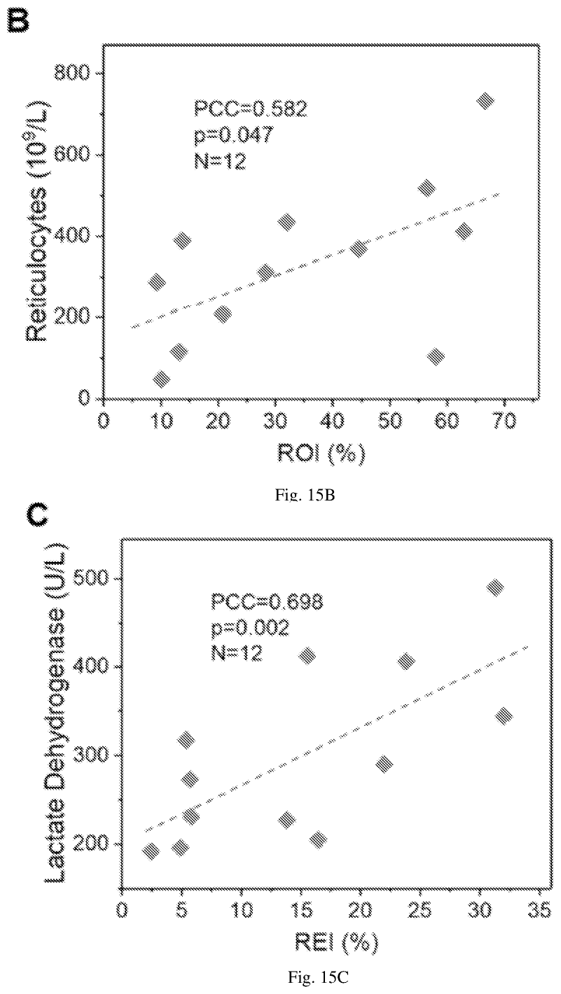

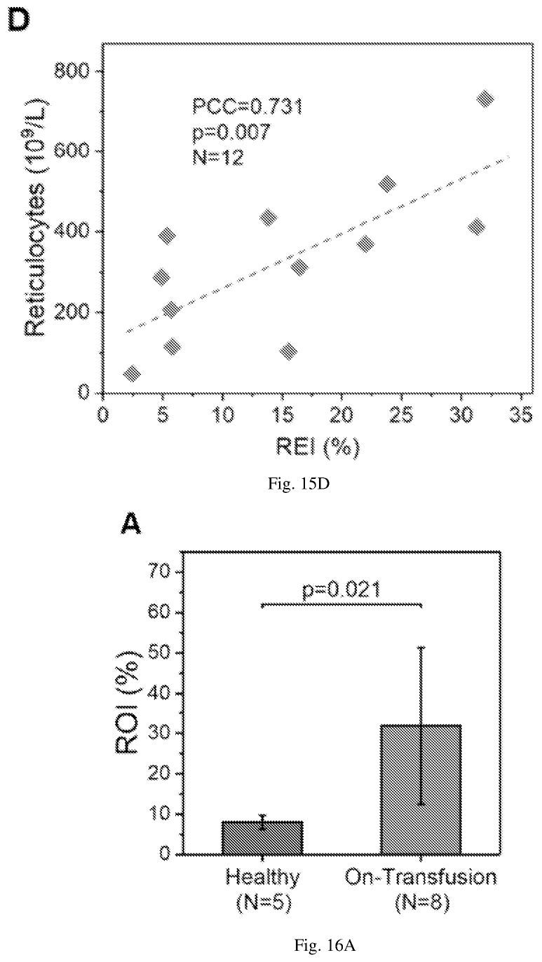

We next explored whether the clinical phenotypes of the study population with SCD affected microvascular occlusion. We found that ROI and REI significantly associated with in vivo biomarkers of hemolysis, including serum LDH levels (FIG. 15A, PCC=0.814, p=0.001) and ARCs (FIG. 15B, PCC=0.582, p=0.047) in the study population with SCD. Next, we assessed whether the electrical impedance change is associated with these biomarkers. Our results indicate that the REI significantly correlates with serum LDH levels (FIG. 15C, PCC=0.698, p=0.012) and ARCs (FIG. 15D, PCC=0.731, p=0.007) in the study subjects with SCD.

### Roi and REI as an In Vitro Therapeutic Benchmark to Assess Clinical Outcome of Treatments in SCD

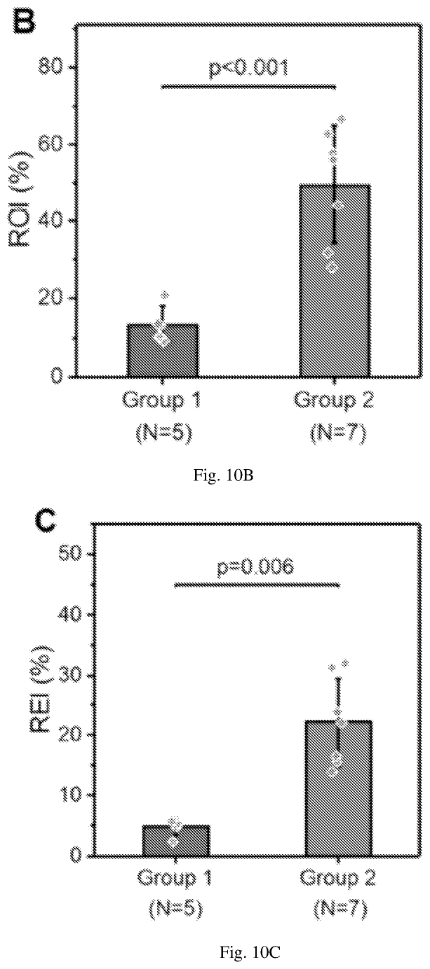

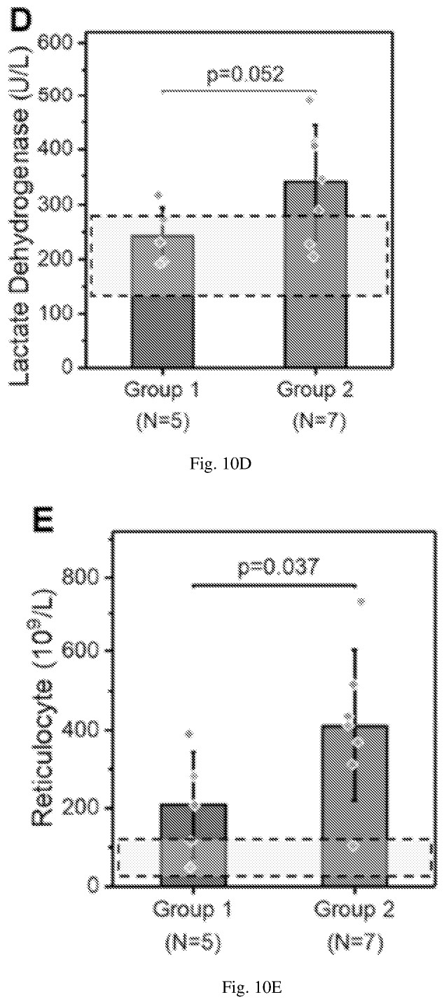

We further assessed whether treatments of the study population with SCD affected microvascular occlusion. We performed mean-shift clustering analysis and identified a sub-group of subjects (Group 1, N=5) with distinct ROI and REI profiles compared to the rest (Group 2, N=7, FIG. 10A). We found that Group 1 subjects had significantly lower levels of ROI (FIG. 10B, mean±SD=13.42±4.57% vs. 49.81±15.13%, p<0.001, one-way ANOVA) and REI (FIG. 10C, mean±SD=4.84±1.39% vs. 22.13±7.40%, p=0.006, Mann-Whitney) compared to Group 2 subjects. We then determined that among the five Group 1 subjects who had less severe microvascular occlusion, one received allogeneic hematopoietic stem-cell transplantation (HSCT), one was on-hydroxyurea (HU), and the other three were on-transfusion (FIG. 10A). We next explored whether the two groups of subjects differ in terms of other clinical variables. Accordingly, we found that Group 1 subjects had relatively lower serum LDH levels (FIG. 10D, 242±53 vs. 339±105 U/L, p=0.052, Mann-Whitney) and significantly higher ARC levels (FIG. 10E, mean±SD=209±136 vs. 412±192 10⁹/L, p=0.037. Mann-Whitney) compared to Group 2 subjects. Comparison of clinical variables between the two groups is summarized in Table 2.

TABLE 2
Comparison of clinical variable between Group 1 and Group 2 subjects in SCD

| | Group 1 (N = 5) | Group 2 (N = 7) | |
|---|---|---|---|
| Clinical variables | Mean ± SD | Mean ± SD | P-value |
| ROI (%) | 13.42 ± 4.57 | 49.81 ± 15.13 | <0.001* |
| REI (%) | 4.84 ± 1.39 | 22.13 ± 7.40 | 0.006** |
| Age (years) | 39 ± 9 | 35 ± 12 | 0.519* |
| Hemoglobin (g/dL) | 9.3 ± 2.2 | 8.0 ± 1.1 | 0.200* |
| Hematocrit (%) | 27.8 ± 6.4 | 23.9 ± 3.6 | 0.203* |
| White Blood Cell Count (10⁹/L) | 12.1 ± 2.5 | 12.7 ± 2.2 | 0.754* |
| Platelet Count (10⁹/L) | 270 ± 145 | 400 ± 123 | 0.125* |
| Absolute Neutrophil Count (10⁶/L) | 7666 ± 1760 | 7426 ± 3413 | 0.889* |
| Absolute Reticulocyte Count (10⁹/L) | 209 ± 136 | 412 ± 192 | 0.037** |
| Lactate Dehydrogenase (U/L) | 242 ± 53 | 339 ± 105 | 0.052** |
| Ferritin (μg/L) | 4109 ± 3050 | 2413 ± 1666 | 0.240* |
| Hemoglobin S (%) | 44.4 ± 27.4 | 44.2 ± 24.9 | 0.992* |
| Hemoglobin A (%) | 42.8 ± 25.8 | 42.3 ± 21.3 | 0.971* |
| Hemoglobin F (%) | 2.6 ± 4.2 | 2.9 ± 2.2 | 0.223** |

ROI: RBC Occlusion Index.
REI: RBC Electrical Impedance Index.
SD: Standard deviation.
*one-way ANOVA
**Mann-Whitney

Results presented in this example show that the microfluidic device and the integrated electrical impedance measurement provide a functional, reproducible in vitro approach for assessing microvascular occlusion associated with abnormal RBC deformability. Glutaraldehyde is a non-specific protein cross-linker, which is commonly used to verify new RBC deformability measurement techniques. Our results on glutaraldehyde-stiffened RBCs suggest that the present microfluidic assay is deformability-based and is able to discriminate different RBC samples with 1% variation in the fraction of stiff RBCs over the entire RBC population (FIG. 7B & D). However, the RBC deformability resulting from glutaraldehyde stiffening is not comparable to that of pathological RBCs in red cell disorders. Therefore, we further validated the microfluidic device with samples from people with RBC disorders, namely, SCD and HS. HS, mostly prevalent among northern Europeans, results in a fragile RBC membrane. We found that RBCs in two subjects with HS were less deformable, as reflected by a higher ROI and REI compared to healthy RBCs (FIG. 9B & D). SCD, prevalent in the African diaspora, is one of the most common inherited blood disorders worldwide and affects millions of people with considerable morbidity and mortality. Both the abnormal RBC deformability and the molecular basis of SCD are well established in SCD. In accordance with previous studies, we found that the ROI and REI of RBCs from people with SCD were significantly higher, therefore less deformable, compared to RBCs from control subjects (FIG. 9B & D). Interestingly, our results show that RBCs from people with SCD had relatively higher ROI and REI compared to those from the two subjects with HS (FIG. 9B & D). An early study using isotonic ektacytometry revealed that reduction in isotonic RBC deformability is higher in SCD than in HS. We postulate that these observations are due to the fact that pathological RBCs from different diseases are affected to different extents.

The microfluidic device described herein has a downstream micropillar array with narrow microcapillaries (3 μm). This feature increases the sensitivity of this test for disease in which a subtle fraction of RBCs are abnormal. Flow rate is a key parameter that could affect the measurement. The flow rate here is mediated by inlet pressure, medium viscosity (dominated by hematocrit), and microchannel flow resistance. For standardized microfluidic assessment, we employed a digital microfluidic pressure pump to allow a constant inlet pressure, and also carefully adjusted the hematocrit value of RBC suspension at 20% for all the tested samples. Further, the microchannel is 12-μm thick, which is larger than the thickness of RBCs (˜ 2 μm). Hence, when RBCs are retained in the microcapillaries, other cells are still able to transit through the microcapillaries. This feature, along with the large scale of the micropillar arrays and the two 40-μm-wide side passageways (anastomoses), make the microfluidic system hard to saturate, largely preventing a potential build-up of flow resistance in the microchannel due to RBC retention. Moreover, the total volume of the blood sample processed within one testing cycle was measured as approximately 40 μL, which translates to a processing rate of 4 million RBCs/min (with an estimated blood density of 1.06 g/mL and 2 million RBCs/μL of blood at 20% hematocrit). Such high volume-processing throughput of our device ensures meaningful test results with clinical blood samples.

The microfluidic device was integrated with surface electrodes for electrical impedance measurement capabilities, and the REI measurements significantly correlate with ROI measurements (FIG. 7E and FIG. 8E), suggesting that REI could serve as a robust indicator of the overall microcapillary occlusion across the device, and that the integrated electrical impedance measurement could replace high-resolution imaging when applying the device in primary healthcare settings. The microfluidic device provides a finer and more rapid detection compared to previous devices, an alternative functional metric for standardized assessments of RBC mediated microvascular occlusion with no need for high-resolution imaging, with the potential to be developed as a truly small-sized, portable device providing real-time results at the point-of-care.

SCD is a clinically heterogeneous disease, as the clinical phenotypes vary considerably from subject to subject. Here, our results show that both the ROI and REI significantly correlate with two in vivo hemolytic biomarkers, serum LDH levels and ARCs (FIG. 15), suggesting that subjects with a more severe intravascular hemolysis are more likely to have RBC-driven microvascular occlusion in SCD. We postulate that two factors may have contributed to these results. Firstly, sickle RBCs have been shown to be vulnerable to mechanical stress and microvesicles shedding-off, which leads to increased hemolysis, reduced membrane surface area-to-volume ratio, and decreased deformability. Secondly, reticulocytes are known to be less deformable compared to mature RBCs, due to their spherical shape with smaller surface area-to-volume ratio, less optimal organization of membrane lipids and proteins, and more viscous cytoplasm with a mass of chromatin granules. The presence of reticulocytosis in a subset of people with SCD, due to hemolytic stress, may contribute to the elevated microcapillary occlusion seen in these studies.

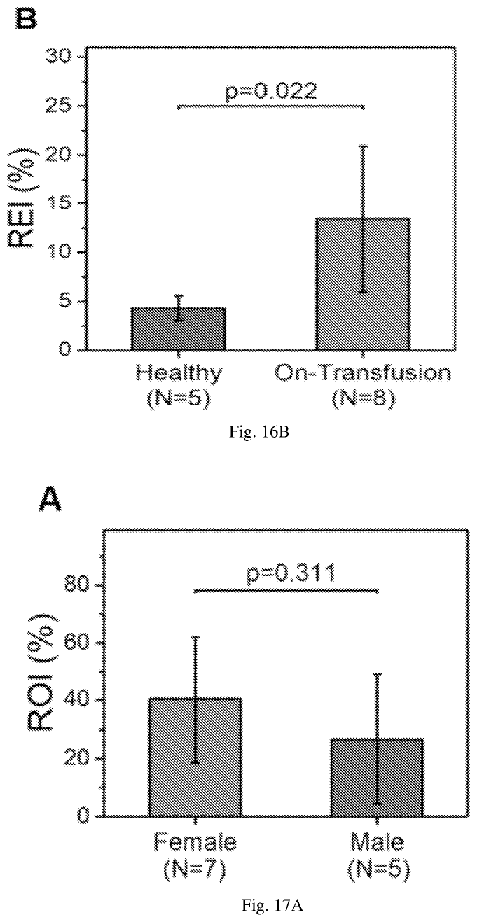

Of note, we observed a cluster of subjects who had significantly lower levels of ROI and REI compared to the rest (FIG. 10A, B & C) over the study population with SCD. Among these five subjects, one received Allogeneic HSCT, one was on-hydroxyurea (HU), and the other three were on exchange transfusion (FIG. 10A). We determined that these subjects had less severe RBC mediated microvascular occlusion as measured by the microfluidic system, which was consistent with their relatively lower serum LDH levels and significantly lower ARCs (FIG. 10D & E). Exchange transfusion is one of the main therapeutic treatments in SCD, in which normal RBCs from healthy donors are exchanged with sickle RBCs to dilute the concentration of sickle hemoglobin and sickle RBCs in circulation. However, vaso-occlusive events can still occur in transfused patients due to the remaining and newly made sickle, non-deformable RBCs. Accordingly, we found that the 8 subjects on transfusion therapy among the study population with SCD had significantly higher ROI and REI compared to healthy donors (FIG. 16). Notably, we did not notice any significant difference when comparing ROI and REI between females and males over the study population (FIG. 17). Together, these analyses demonstrate the promise of the microfluidic assay and the REI as an in vitro therapeutic efficacy benchmark to assess clinical outcomes. These tests are likely to significantly benefit the development and assessment of targeted and curative therapies for SCD.

Therapeutic ex vivo gene transfer into autologous hematopoietic stem cells, also known as gene therapy, is currently the most promising approach that can repair the fundamental cause of SCD and provide long-term curative treatment for the patients. The National Heart, Lung, and Blood Institute recently launched the ‘Cure Sickle Cell Initiative’ to keep nourishing the collaborative, patient-focused research environment and to support the further development of gene therapy for treating SCD. Close monitoring of the ability of RBCs to clear microcapillaries in the presented microfluidic device before and after a curative therapy would provide valuable insights into the patient clinical outcome. In particular, discrepant RBC populations may arise in the patient after receiving a curative therapy, at which time it is crucial to discern the heterogeneity in the entire cell population and its effects. In this study, we found that one patient with SCD, who received Allogeneic HSCT (FIG. 10A), had similar levels of ROI and REI compared to healthy donors (ROI: 10.08% vs. 8.02±1.71%; REI: 2.44% vs. 4.31±1.25%). Based on our results, we will test whether, following curative therapy, we will see a transition from high to low microcapillary occlusion and electrical impedance as non-sickling RBCs replace sickle RBCs in vivo.

SARS-COV-2, a new RNA coronavirus leading to a global pandemic, is the etiological driver of the clinical syndrome coronavirus disease 2019 (COVID-19). The disease is characterized by a number of manifestations including persistent dry cough, shortness of breath, hypoxemia, and fever. In SCD, COVID-19 may trigger severe acute chest syndrome (ACS) and vaso-occlusive crisis. Since RBCs play an important role in oxygen delivery, it is plausible to suspect that their biophysical properties, such as deformability, are deleteriously altered and thus contribute to congestions of microvessels in COVID-19. Importantly, a recent study showed that COVID-19 is linked with significantly less deformable RBCs, even though the underlying mechanism is yet to be uncovered. Within the context of COVID-19, we envision that our microfluidic assay and the REI may provide a functional biomarker to supplement the current diagnostic tools for disease assessment and monitoring.

Unique features of the present microfluidic device include processing of clinical blood samples at near-physiologic hematocrit (20%), rapid (less than 5 min) data acquisition and analysis, and REI as an alternative functional metric for occlusion assessment in the absence of high-resolution imaging and as a functional test for microvascular health and disease status monitoring.

### Example 2

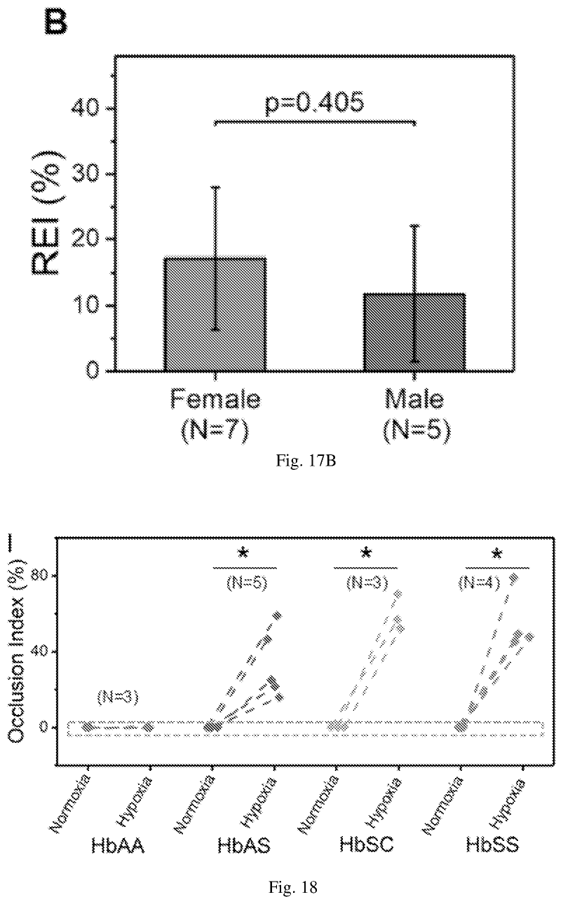

Abnormal RBC deformability contributes to the underlying pathophysiology of SCD, and we tested this in variant SCD and sickle cell trait (SCT). We have developed MIRCA, a novel functional assay of RBC deformability, assessed by quantitative microcapillary occlusion (‘Occlusion Index, OI’, FIG. 18). This in vitro test recapitulates key geometric features of the capillary bed: >80,000 narrow openings and side anastomoses ‘on-a-chip’, under normoxia or hypoxia (FIG. 18).

In people with variant SCD (HbSC) and SCA (HbSS), RBC OI is significantly higher. OI inversely associated with hydroxyurea use and % HbF and was exaggerated under hypoxia proportionate with % HbS. In this task we will test whether cellular, biophysical, and rheological aberrations are present in variant SCD and SCT, under normoxia and hypoxia in vitro (FIG. 18).

Variant SCD has a milder clinical phenotype overall, but nonetheless confers significant morbidity, suffering, and unexpected early mortality. RBCs from people with variant disease show significant cellular and rheological abnormalities (FIG. 18), as reported by us and others. However, cellular physiology in variant disease is rarely studied.

More than 3 million Americans (8-10% of African Americans), and 300 million people across the globe, live with SCT (HbAS). Despite normal blood counts and little reported subjective impact, people with SCT may be at risk for hematuria, thrombophilia, renal disease, atrial fibrillation, and (rarely) exertional rhabdomyolysis. In light of this clinical paradox, the National Academies recommend that the NIH support research on the impact of SCT.

From the above description of the invention, those skilled in the art will perceive improvements, changes and modifications. Such improvements, changes and modifications within the skill of the art are intended to be covered by the appended claims. All references, publications, and patents cited in the present application are herein incorporated by reference in their entirety.

The invention claimed is:

1. A microfluidic device comprising:
at least one microchannel that extends through a portion of a housing, the at least one microchannel being configured to receive a fluid sample that flows along a length of the microchannel from a first end to a second end of the microchannel, the at least one microchannel including a plurality of micropillar arrays provided along the length of the microchannel and pairs of electrodes positioned on opposite sides of each micropillar array in a direction of fluid flow through the microchannel, wherein each micropillar array defines a plurality of microcapillaries having a separation distance and the separation distance of the microcapillaries defined by each micropillar array decreases in a direction of fluid flow through the microchannel, and wherein the pairs of electrodes are configured to measure electrical impedance in each respective micropillar array.

2. The microfluidic device of claim 1, the microchannel including a substantially planar upper surface and a substantially planar lower surface, the micropillars of the plurality of micropillar arrays extending from upper surface to the lower surface.

3. The microfluidic device of claim 1, wherein each of the micropillars of the plurality of micropillar arrays has a substantially rectangular cross section.

4. The microfluidic device of claim 1, wherein each of the microcapillaries has a substantially rectangular cross section.

5. The microfluidic device of claim 1, wherein the microchannel includes at least three micropillar arrays and the cross sectional areas of the microcapillaries defined by each respective micropillar array being substantially uniform.

6. The microfluidic device of claim 1, wherein each micropillar array includes at least three rows of micropillars, the rows extending perpendicular to fluid flow and having a substantially similar shape.

7. The microfluidic device of claim 1, wherein the distance between each micropillar in a row of a respective micropillar array is substantially the same.

8. The microfluidic device of claim 1, wherein successive micropillar arrays are separated from each other in the microchannel by a gap region, the gap region being free of micropillars and wherein each gap region includes an electrode.

9. The microfluidic device of claim 1, wherein the microchannel includes a first micropillar array at the first end that defines a plurality of first microcapillaries that each have a separation distance of about 12 μm and each successive micropillar array in the direction of fluid flow through the microchannel defines a plurality of microcapillaries that each have a separation distance about 10% to about 30% less than a plurality of microcapillaries defined by a preceding micropillar array.

10. The microfluidic device of claim 1, wherein the microchannel includes a micropillar array at the second end that defines a plurality of microcapillaries that each have a separation distance of about 3 μm and each preceding micropillar array in a direction opposite the direction of fluid flow through the microchannel defines a plurality of microcapillaries that each have a separation distance about 20% to about 50% greater than a plurality of microcapillaries defined by a preceding micropillar array.

11. The microfluidic device of claim 1, wherein the separation distance of the plurality of microcapillaries defined by at least one of the plurality of micropillar arrays permits passage of healthy cells in a fluid sample perfused through the microchannel but occludes cells with impaired deformability.

12. The microfluidic device of claim 1, wherein the fluid sample includes blood cells.

13. (canceled)

14. The microfluidic device of claim 1, wherein the separation distance of the plurality of microcapillaries at the second end of the microchannel occludes abnormal red blood cells in a fluid sample perfused through the microchannel.

15. The microfluidic device of claim 1, wherein each of the micropillar arrays is arranged in an inner portion of the microchannel that extends the length of the microchannel, the microchannel including two parallel outer passages on opposite sides of the inner portion that extend the length of the microchannel, the outer passages being in fluid communication with the plurality of microcapillaries defined by the plurality of micropillar arrays.

16. The microfluidic device of claim 1, wherein the outer passages have cross sectional areas that permit cells in a fluid sample to flow through the microchannel without being occluded and/or obstructed.

17. The microfluidic device of claim 1, wherein the electrodes are planar and are provided on a lower surface channel of the microchannel.

18. The microfluidic device of claim 1, wherein the measured impedance is indicative of red blood cell mediated occlusion in the respective micropillar array.

19. The microfluidic device of claim 1, wherein the microchannel includes a substantially planar transparent wall that defines the upper surface or lower surface of the microchannel.

20. The microfluidic device of claim 19, wherein the substantially planar transparent wall permits observation into the microfluidic channel by microscopy.

21. The microfluidic device of claim 1, further comprising a micro-gas exchanger and a chamber for controlling the oxygen content of the fluid sample prior to and/or after delivering the fluid sample to the at least one microchannel.

22. The microfluidic device of claim 21, the micro-gas exchanger and the chamber providing hypoxic blood to the at least one microchannel.

23. The microfluidic device of claim 1, further comprising at least one capturing agent that is immobilized on a surface of the at least one microchannel, the capturing agent adhering a cell of interest to the at least one surface of the at least one microchannel when a fluid sample containing cells is passed through the at least one microchannel.

24. The microfluidic device of claim 23, the at least one capturing agent comprising at least one of laminin, fibronectin, E-Selectin, P-Selectin, L-selectin, intracellular adhesion molecule 1 (ICAM-1), or vascular cellular adhesion molecule 1 (VCAM-1).

25-59. (canceled)
